(19) United States
(12) Patent Application Publication
(10) Pub. No.: US 2025/0144217 A1
(43) Pub. Date: May 8, 2025

(54) LOCALLY ADMINISTERED COMPOSITIONS AND METHODS OF USE THEREOF

(71) Applicants: FAIRLEIGH DICKINSON UNIVERSITY, Teaneck, NJ (US); ONCOGONE, INC., Frederick, MD (US)

(72) Inventors: Hyunah CHO, Cedar Grove, NJ (US); Xiaoban XIN, Frederick, MD (US)

(21) Appl. No.: 18/837,038

(22) PCT Filed: Feb. 10, 2023
(86) PCT No.: PCT/US2023/012812

§ 371 Date: Aug. 8, 2024

Related U.S. Application Data
(60) Provisional application No. 63/309,125, filed on Feb. 11, 2022.

(51) Int. Cl. A61K 47/34; A61K 9/06; A61K 31/337; A61K 31/495

(52) U.S. Cl. CPC A61K 47/34; A61K 9/06; A61K 31/337; A61K 31/495

(57) ABSTRACT

A composition includes a first polymer, a second polymer, and one or more therapeutic agents. The first and the second polymers are PLGA-PEG-PLGA triblock copolymers. The first polymer has a molecular weight higher than the second polymer; a molar ratio of the first polymer to the second polymer is from 3:1 to 1:1; and a ratio of a weight of the one or more therapeutic agents to a total weight of the first and second polymers is at least 2%.

### CROSS-REFERENCE TO RELATED APPLICATIONS

This application claims the benefit of U.S. Provisional Application No. 63/309,125, filed Feb. 11, 2022. The entire contents of the above-identified application are hereby fully incorporated herein by reference.

### TECHNICAL FIELD

The subject matter disclosed herein is generally directed to hydrogel formulations and methods of use thereof.

### BACKGROUND

Hydrogels have emerged as a safe and effective drug delivery systems for treatment various diseases, e.g., cancers. However, hydrogels have been limited to carrying hydrophilic drugs rather than hydrophobic drugs. Being able to deliver hydrophobic drugs via hydrogel-based delivery systems is important, e.g., for locally administered treatment such as chemotherapy. A large number of chemotherapeutic drug candidates possess poor water solubility and have failed to reach sufficient concentrations in injectable solutions required to exhibit therapeutic potency in preclinical studies and clinical trials. Incorporation of poorly water-soluble drugs into hydrogels can enhance aqueous solubility of drugs and achieve extended release of drugs thereby increasing chances of intratumoral uptake of drugs than free drugs.

Accordingly, there is a need to develop novel hydrogel formulations capable of delivering sufficient amount of therapeutics agents with various physical and chemical features.

### SUMMARY

In an aspect, the present disclosure provides a composition comprising: a first polymer and a second polymer; and one or more therapeutic agents, wherein: the first and the second polymers are PLGA-PEG-PLGA triblock copolymers, the first polymer has a molecular weight higher than the second polymer, a molar ratio of the first polymer to the second polymer is from 3:1 to 1:1, and a ratio of a total weight of the one or more therapeutic agents to a total weight of the first and second polymers is at least 2%. In some embodiments, the ratio of a total weight of the one or more therapeutic agents to a total weight of the first and second polymers is at least 30%.

In some embodiments, the ratio of the total weight of the one or more therapeutic agents to the total weight of the first and second polymers is from 30% to 80%. In some embodiments, the ratio of the total weight of the one or more therapeutic agents to the total weight of the first and second polymers is from 45% to 65%. In some embodiments, the ratio of the weight of the one or more therapeutic agents to the total weight of the first and the second polymers is from 58% to 62%. In some embodiments, the ratio of the weight of the one or more therapeutic agents to the total weight of the first and the second polymers is at least 90%. In some embodiments, the ratio of the weight of the one or more therapeutic agents to the total weight of the first and the second polymers is from 30% to 145%.

In some embodiments, the composition further comprises water, wherein a ratio of a weight of the water to a total weight of other components in the composition is from 80:1 to 15:1. In some embodiments, the ratio of a weight of the water to a total weight of other components in the composition is 70:1 to 20:1. In some embodiments, the molar ratio between the first and the second polymers is about 2:1.

In some embodiments, the composition is a hydrogel. In some embodiments, the composition is a non-flowing thermosensitive hydrogel at a temperature from 28° C. to 50° C. and a free-flowing solution at a temperature from 0° C. to 26° C. In some embodiments, the composition is in a solid form, and the solid form becomes a hydrogel when mixing with water or an aqueous solution from about 20% g/mL to 70% g/mL measured as total weight of the solid form by volume of water. In some embodiments, the composition further comprises a bulking agent. In some embodiments, a ratio of the total weight of the first polymer, the second polymer and the therapeutic agents to a weight of the bulking agent is at least 9. In some embodiments, the bulking agent is mannitol.

In some embodiments, the one or more therapeutic agents comprises one or more chemotherapeutic drugs. In some embodiments, the one or more chemotherapeutic drugs are hydrophobic. In some embodiments, the one or more chemotherapeutic drugs comprises a taxane compound. In some embodiments, the taxane compound is paclitaxel or an analog thereof. In some embodiments, a ratio of a weight of the paclitaxel or an analog thereof to the total weight of the first and the second polymers is from 1.5% to 5%. In some embodiments, a ratio of a weight of the paclitaxel or an analog thereof to the total weight of the first and the second polymers is from 1.5% to 43%. In some embodiments, the one or more chemotherapeutic drugs comprises an imidazotetrazine derivative compound. In some embodiments, the imidazotetrazine derivative compound is temozolomide or an analog thereof. In some embodiments, a ratio of a weight of the temozolomide or an analog thereof to the total weight of the first and the second polymers is from 30% to 70%. In some embodiments, a ratio of a weight of the temozolomide or an analog thereof to the total weight of the first and the second polymers is from 30% to 100%. In some embodiments, the composition comprises two therapeutic agents. In some embodiments, the composition comprises two chemotherapeutic drugs. In some embodiments, the two chemotherapeutic drugs comprise an imidazotetrazine derivative compound and a taxane compound. In some embodiments, the one or more chemotherapeutic drugs comprises paclitaxel or an analog thereof, and temozolomide or an analog thereof. In some embodiments, a ratio of the total weight of a weight of the paclitaxel or analog thereof to the total weight of the first and the second polymers is from 1.5% to 5%, and a ratio of a weight of the temozolomide or analog thereof to the total weight of the first and the second polymers is 30% to 70%. In some embodiments, a. a ratio of the total weight of a weight of the paclitaxel or analog thereof to the total weight of the first and the second polymers is from 1.5% to 43%, and a ratio of a weight of the temozolomide or analog thereof to the total weight of the first and the second polymers is 30% to 100%.

In some embodiments, the composition is in a form of powder. In some embodiments, the composition is in a form of a pellet. In some embodiments, the pellet has a weight from 3 mg to 10 mg. In some embodiments, the pellet has a weight from 5 mg to 7 mg. In some embodiments, the composition further comprises an ointment base.

In another aspect, the present disclosure provides a method of treating a disease, the method comprising administering the composition according to the present disclosure to a subject in need thereof.

In some embodiments, the disease is cancer. In some embodiments, the cancer is glioblastoma or astrocytoma. In some embodiments, the cancer is melanoma. In some embodiments, the cancer is Kaposi sarcoma, breast cancer, lung cancer, ovarian cancer, or adenocarcinoma. In some embodiments, the cancer is angiosarcoma, bladder cancer, esophagus cancer, prostate cancer, cervical cancer, gastric cancer, head and neck cancer, lymphoma, neoplasm of endometrium of corpus uteri, nasopharynx cancer, myeloma, small cell lung cancer, or testicular cancer.

In some embodiments, the composition is administered locally. In some embodiments, the composition is administered intracranially. In some embodiments, the composition is administered topically. In some embodiments, the amount of one or more therapeutic agents administered to the subject would cause systemic toxicity if administered systemically, and wherein the administration causes decreased or no systemic toxicity.

In another aspect, the present disclosure provides a method of producing a composition comprising: (a) mixing a first polymer, a second polymer, and one or more therapeutic agents in water; (b) mixing an organic solvent with the mixture from (a); and (c) lyophilizing the mixture from (b), wherein the first and the second polymers are PLGA-PEG-PLGA triblock copolymers, the first polymer has a molecular weight higher than the second polymer, a molar ratio of the first polymer to the second polymer is from 3:1 to 1:1, and a ratio of a weight of the one or more therapeutic agents to a total weight of the first and second polymers is at least 30%.

In some embodiments, (a) comprises mixing a solution of the first polymer, a solution of the second polymer, and the one or more therapeutic agents. In some embodiments, the one or more therapeutic agent is temozolomide. In some embodiments, (a) comprises mixing the first polymer, the second polymer, and a first therapeutic agent in water, and (b) comprises mixing a second therapeutic agent with the organic solvent with the mixture of (a). In some embodiments, (a) comprises mixing the first polymer, the second polymer, a bulking agent. and a first therapeutic agent in water, and (b) comprises mixing a second therapeutic agent with the organic solvent with the mixture of (a). In some embodiments, the bulking agent is mannitol. In some embodiments, the first therapeutic agent is temozolomide and the second therapeutic agent is paclitaxel. In some embodiments, the organic solvent is acetone. In some embodiments, the method further comprises freeze-drying the mixture of (b) prior to (c).

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-C—An exemplary composition comprising mannitol (10 mg)/paclitaxel (2 mg)/temozolomide (30 mg) in the form of lyophilized gel powder (FIG. 1A) and the sol phase (gel powder reconstituted with 200 mcL of water) (FIGS. 1B and 1C) at 20° C. FIGS. 1D-1F show an exemplary composition comprising mannitol (10 mg)/temozolomide (30 mg) in the form of lyophilized gel powder (FIG. 1D) and the sol phase (gel powder reconstituted with 200 mcL of water) (FIGS. 1E and 1F) at 20° C.

FIG. 2A shows an exemplary composition comprising mannitol (10 mg)/paclitaxel (2 mg)/temozolomide (30 mg) in the gel phase at 37° C. FIG. 2B shows the exemplary composition comprising mannitol (10 mg)/paclitaxel (2 mg)/temozolomide (30 mg) in the form of lyophilized gel powder pressed into a solid pellet. FIG. 2C shows an exemplary composition comprising mannitol (10 mg)/temozolomide (30 mg) in the gel phase at 37° C. FIG. 2D shows the exemplary composition comprising mannitol (10 mg)/temozolomide (30 mg) in the form of lyophilized gel powder pressed into a solid pellet.

FIGS. 3A-3B show an exemplary composition comprising mannitol (10 mg)/paclitaxel (2 mg)/temozolomide (30 mg) in the gel form (FIG. 3A) and the form of solid pellet (FIG. 3B) adherent to glass scintillation vials. FIGS. 3C-3D show an exemplary composition comprising mannitol (10 mg)/temozolomide (30 mg) in the gel form (FIG. 3C) and the form of solid pellet (FIG. 3D) adherent to glass scintillation vials.

FIGS. 4A-4B show that no gelation occurred with PLGA-PEG-PLGA triblock (1.5k-1.5K-1.5K) polymer alone in water at 37° C. (left) and gelation occurred with PLGA-PEG-PLGA triblock (1k-1K-1K) alone in water at 20° C. (right).

FIG. 5 shows drug release profile of an exemplary composition comprising mannitol (10 mg)/paclitaxel (2 mg)/temozolomide (30 mg) at 37° C.

FIG. 6 shows drug release profile of an exemplary composition comprising mannitol (10 mg)/temozolomide (30 mg) at 37° C.

FIG. 7 shows the inhibitory effects of the TMZ hydrogel cream and TMZ/PTX hydrogel cream on xenograft melanoma.

FIG. 8A shows the body weights of the animals during the experiment in Example 7. FIG. 8B shows the effect of hydrogel carrying TMZ on the survival of animals of glioblastoma model.

The figures herein are for illustrative purposes only and are not necessarily drawn to scale.

### DETAILED DESCRIPTION OF THE EXAMPLE EMBODIMENTS

### Definitions

As used herein, the singular forms “a”, “an”, and “the” include both singular and plural referents unless the context clearly dictates otherwise.

The recitation of numerical ranges by endpoints includes all numbers and fractions subsumed within the respective ranges, as well as the recited endpoints.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The term “therapeutic agent,” which is used interchangeably with “drug,” herein, refers to any pharmacologically active substance capable of being administered which achieves a desired effect. Such agents can be synthetic or naturally occurring, small molecule compounds (e.g., chemotherapeutic drugs), non-peptide, proteins or peptides, oligonucleotides or nucleotides, polysaccharides or sugars.

A “hydrophobic therapeutic agent” or a “hydrophobic drug” refers to a therapeutic agent that has poor water solubility. Such therapeutic agents may have a solubility of less than 10 mg/mL in distilled water at 25° C. A hydrophobic therapeutic agent may have a solubility of about 1-10 mg/mL or even 0.1-1 mg/mL. These terms are well-known to those of skill in the art. See, e.g., Martin (ed.), Physical Pharmacy, Fourth Edition, page 213 (Lea and Febiger 1993). When hydrophobic drugs can be successfully used to prepare stable hydrogel formulations, they can be extruded through a 22-gauge needle at below their gelation temperature. Thus, these compositions can be suitable for local treatments using a hydrophobic drug or combination of hydrophobic drugs, as described herein.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides compositions carrying one or more therapeutic agents (e.g., chemotherapeutic drugs). In one aspect, the composition may be a hydrogel carrying the one or more therapeutic agents (e.g., chemotherapeutic drugs). At least one of the therapeutic agents may be hydrophobic. In some examples all the therapeutic agent(s) are hydrophobic. In some embodiments, the composition may be administered to a subject locally, e.g., to a location not readily reachable by the drugs if administered systemically, and/or to achieve a higher local concentration of the drugs compared to other administration route (e.g., systemic administration). Compared to systemic administration, such local treatment may enhance the efficacy of the drugs (e.g., due to higher local drug concentration) and/or reduce toxicity to other tissues (e.g., reduce or minimize drug concentration in circulation and other tissues). In some embodiments, the hydrogel compositions may be administered to the brain for delivering drugs that have poor penetration through the blood brain barrier (BBB) if administered systemically. For example, the hydrogel compositions may be administered to the brain for treating brain tumors, e.g., glioblastoma.

The composition may carry multiple therapeutic agents, e.g., multiple hydrophobic therapeutic agents. In some examples, after administration, two therapeutic agents may be released from the composition at different rates. The therapeutic agent released first may be a sensitizer of the therapeutic agent released later, thereby providing synergistic therapeutic effects.

In some embodiments, the composition herein may be thermosensitive. For example, the composition may form a stable non-flowing gel at or above a gelation temperature (“gel phase”) and is or starts to transition to a free-flowing solution below the gelation temperature (“sol phase”). In both the gel and sol phases, the hydrogel composition may incorporate one or more therapeutic agents. In some embodiments, the gelation temperature may be above the room temperature (e.g., about 25° C.) and below the body temperature (e.g., about 37° C.). This is advantageous compared to conventional hydrogel compositions that have a gelation temperature at or below the room temperature. With such low gelation temperature, the conventional hydrogel compositions may have been already in the gel phase in an injection device (e.g., syringe) before reaching the administration site in a subject, which may make the administration process cumbersome and cause pain and other adverse effect in the subject. In contrast, the higher gelation temperature of the composition according to the present disclosure may allow easy administration of the composition with an injection device (e.g., a syringe) in its free-flowing sol phase. After being injected to the subject, the composition may transition into the gel phase, which reduces the pain and other adverse effects due to cumbersome administration procedures.

In some embodiments, the composition may comprise a first polymer and a second polymer; and one or more therapeutic agents, wherein: the first and the second polymers are PLGA-PEG-PLGA triblock copolymers, the first polymer has a molecular weight higher than the second polymer, a molar ratio of the first polymer to the second polymer is from 3:1 to 1:1, and the ratio of the weight of the one or more therapeutic agents to a total weight of the first and second polymers is above 40%. The inventors have surprisingly found that the compositions have the thermosensitivity feature with higher gelation temperature and are also capable of carrying and delivering a high amount of hydrophobic therapeutic agent(s).

Also provided herein are kits and devices comprising the compositions as well as methods of using and making the compositions.

### Compositions

In one aspect, the present disclosure provides compositions carrying one or more therapeutic agents to a targeted site of a subject. In some embodiments, the compositions may comprise two polymers and one or more therapeutic agents. The composition may be administered to a targeted site in a subject to provide sustainable release of the one or more therapeutic agents at the site.

The composition may be formulated in a way that different therapeutic agents are released at different rates. Such different rates may provide improved therapeutic effects. For example, the first released therapeutic agent may sensitize the tissue or cell at and/or around the administration site to respond to the later-released therapeutic agent. In such cases, the compositions may provide a synergistic effect of multiple therapeutic agents. In some examples, the release of the therapeutic agents may depend on their hydrophilicity. For example, a composition comprising temozolomide and paclitaxel may release the temozolomide faster than paclitaxel. In some examples, the release of the therapeutic agents may depend on dosage form. For example, therapeutic agents in a solid form composition (e.g., pellet) may be released slower than the same therapeutic agents in a sol or gel phase.

### Polymers

The compositions may comprise a first polymer and a second polymer. The molecular weight of the first polymer may be higher than the molecular weight of the second polymer.

In some embodiments, the first and the second polymers may be ABA-type triblock copolymers, where the A-blocks are a poly(lactide-co-glycolide) and the B-block is a polyethylene glycol (PEG). “PLGA (Poly(lactide-co-glycolide)” or “PLGA” refers to a copolymer derived from the condensation copolymerization of lactic acid and glycolic acid, or by the ring opening polymerization of α-hydroxy acid precursors, such as lactide or glycolide. The terms “lactide” and “lactate” and “glycolide” and “glycolate” can be used interchangeably. In some examples, the A-block segments may be poly(α-hydroxy acids) derived or selected from the group of poly(D,L-lactide-co-glycolide) and poly(L-lactide-co-glycolide), referred to collectively as poly(lactide-co-glycolide).

The compositions may be prepared from the ABA-type block copolymers described herein. The polymers can be ABA-type block copolymers having hydrophobic A block segments and a hydrophilic B block segment. In some examples, the polymers may be ABA-type block copolymers having PLGA A-block segments and PEG B-block segments of the formula: PLGA-PEG-PLGA, wherein the block copolymers that have utility as described herein, namely, compositional make-up within the indicated ranges that result in block copolymers that demonstrate the desired stable thermal gelling behavior.

The hydrophobic A-blocks may have biodegradable, biocompatible, and solubilization properties. The in vitro and in vivo degradation of these hydrophobic poly(lactide-co-glycolide) A-blocks is well understood and the degradation products may be naturally occurring compounds that are readily metabolized and/or eliminated by the patient's body. The PEG block may be a hydrophilic, water-soluble block because of its unique biocompatibility, nontoxicity, hydrophilicity, solubilization properties, and rapid clearance from a patient's body. The hydrophilic B-block may be formed from appropriate molecular weights of PEG.

In some embodiments, the average molecular weight (in Daltons) of each poly(lactide-co-glycolide) polymeric A block in the first polymer may be from 700 Da to 1950 Da. For example, the average molecular weight of poly(lactide-co-glycolide) in the polymers may be from 800 Da to 1800 Da, from 1000 Da to 1700 Da, from 1200 Da to 1600 Da, from 1400 Da to 1600 Da, or from 1450 to 1550 Da. In some embodiments, the average molecular weight may be about 800 Da, about 900 Da, about 1000 Da, about 1100, Da, about 1200 Da, about 1300 Da, about 1400 Da, about 1500 Da, about 1600 Da, about 1700 Da, about 1800 Da, or about 1900 Da, or ranges between any two of these values (including endpoints). In an example, the average molecular weight (in Daltons) of each PLGA A block in the first polymer may be about 1500 Da.

In some embodiments, the average molecular weight (in Daltons) of each PEG B block in the first polymer may be from 700 Da to 1950 Da. For example, the average molecular weight of PEG in the polymers may be from 800 Da to 1800 Da, from 1000 Da to 1700 Da, from 1200 Da to 1600 Da, from 1400 Da to 1600 Da, or from 1450 to 1550 Da. In some embodiments the average molecular weight may be about 800 Da, about 900 Da, about 1000 Da, about 1100, Da, about 1200 Da, about 1300 Da, about 1400 Da, about 1500 Da, about 1600 Da, about 1700 Da, about 1800 Da, or about 1900 Da, or ranges between any two of these values (including endpoints). In an example, the average molecular weight (in Daltons) of each PEG block in the first polymer may be about 1500 Da.

In some embodiments, the average molecular weight (in Daltons) of each PLGA A block in the second polymer may be from 600 Da to 1300 Da. For example, the average molecular weight of poly(lactide-co-glycolide) in the polymers may be from 700 Da to 1200 Da, from 800 Da to 1100 Da, from 900 Da to 1050 Da, or from 950 Da to 1050 Da. In some embodiments the average molecular weight may be about 700 Da, about 750 Da, about 800 Da, about 850, Da, about 900 Da, about 950 Da, about 1000 Da, about 1050 Da, about 1100 Da, about 1150 Da, or about 1200 Da, or ranges between any two of these values (including endpoints). In an example, the average molecular weight (in Daltons) of each PLGA A block in the second polymer may be about 1000 Da.

In some embodiments, the average molecular weight (in Daltons) of each PEG B block in the second polymer may be from 600 Da to 1300 Da. For example, the average molecular weight of PEG in the polymers may be from 700 Da to 1200 Da, from 800 Da to 1100 Da, from 900 Da to 1050 Da, or from 950 Da to 1050 Da. In some embodiments the average molecular weight may be about 700 Da, about 750 Da, about 800 Da, about 850, Da, about 900 Da, about 950 Da, about 1000 Da, about 1050 Da, about 1100 Da, about 1150 Da, or about 1200 Da, or ranges between any two of these values (including endpoints). In an example, the average molecular weight (in Daltons) of each PEG B block in the second polymer may be about 1000 Da.

In some examples, the first polymer may be PLGA_{1.5k}-PEG_{1.5k}-PLGA_{1.5k}, in which the PLGA and PEG blocks have a molecular weight of about 1500 Dalton, and the second polymer may be PLGA₁ₖ-PEG₁ₖ-PLGA₁ₖ, in which the PLGA and PEG blocks have a molecular weight of about 1000 Dalton.

For purposes of molecular weight parameters, the molecular weight values may be based on measurements by NMR or GPC (gel permeation chromatography) analytical techniques. The reported weight average molecular weights and number average molecular weights can be determined by GPC and NMR, respectively. The lactide/glycolide ratio can be calculated from NMR data. GPC analysis can be performed, for example, on a Styragel HR-3 column calibrated with PEG using RI detection and chloroform as the eluent. NMR spectra can be taken in CDCl₃. Examples of PLGA-PEG-PLGA triblock copolymers include those commercially available from suppliers such as Polyscitech (West Lafayette, Ind.; www.polyscitech.com).

In the compositions, the first and the second polymers may have a suitable molar ratio that provides the desired features described herein. In some embodiments, the molar ratio of the first to the second polymers in the compositions may be from 10:1 to 1:10, e.g., from 5:1 to 1:5, from 4:1 to 1:4, from 3:1 to 1:3, from 3:1 to 1:2, or from 3:1 to 1:1. In some embodiments, the molar ratio of the first polymer to the second polymer in the composition may be about 5:1, about 4:1, about 3:1, about 2:1, about 1:1, about 1:2, about 1:3, about 1:4, or about 1:5. In one example, the molar ratio of the first polymer to the second polymer in the composition may be about 2:1.

### Therapeutic Agents

The compositions may comprise one or more therapeutic agents. The one or more therapeutic agents may be incorporated into the network formed by the first and the second polymers. In some examples, the therapeutic agent(s) may be small molecule drug(s). In some examples, the therapeutic agent(s) may be chemotherapeutic drug(s). In some embodiment, at least one of the therapeutic agent(s) may be hydrophobic. For example, all the therapeutic agent(s) in the composition may be hydrophobic.

In some embodiments, the one or more therapeutic agents may comprise an imidazotetrazine derivative compound. In one example, the imidazotetrazine derivative compound may be temozolomide. Additional examples of the imidazotetrazine derivative compounds include analogs of temozolomide, e.g., C8-imidazolyl (377) and C8-methylimidazole (465) tetrazines (e.g., as described in Zikuan Yang et al., Front. Oncol., 11 Jun. 2019, doi.org/10.3389/fonc.2019.00485), NE0212 (e.g., as described in Thomas Chen et al., Journal of Biomedical Science volume 22, Article number: 71 (2015)), DP68 and DP86 (e.g., as described in Yulian P. Ramirez et al., Mol Cancer Ther. 2015 January; 14(1):111-9), and analogs described in Catherine L. Moody et al., Pharmaceuticals (Basel). 2014 July; 7(7): 797-838. All the references are incorporated herein by reference in their entireties.

In some embodiments, the one or more therapeutic agents may comprise a taxane compound. In one example, the taxane compound may be paclitaxel. In one example, the taxane compound may be docetaxel. In one example, the taxane compound may be cabazitaxel. Additional examples of taxane compounds include paclitaxel analogs, e.g., docetaxel, cabazitaxel, larotaxel, milataxel, ortataxel, and tesetaxel, BMS-184476, taxoprexin, opaxio, 7-hexanoyltaxol (QP2), 3′-desphenyl-3′-(4-ntirophenyl)-N-dibenzoyl-N-(t-butoxycarbonyl)-10-deacetyltaxol, and other known paclitaxel analogs, e.g., those described in U.S. Pat. No. 5,399,726 (Holton et al.); U.S. Pat. No. 5,470,866 (Kingston et al.); U.S. Pat. No. 5,654,447 (Holton et al.); U.S. Pat. No. 6,107,332 (Ali et al.); U.S. Pat. No. 6,118,011 (Mayhew et al.); and U.S. Pat. No. 6,136,961 (Dordick et al.), all of which are incorporated herein by reference in their entireties.

In some examples, the one or more therapeutic agents may comprise rapamycin or an analog thereof. In one example, the one or more therapeutic agents may comprise rapamycin. In some examples, the one or more therapeutic agents may comprise a rapamycin analog, e.g., AP23573 (ARIAD), CCI779 (“temsirolimus”, Wyeth), RAD001 (“Everolimus”, Novartis), C-43-modified rapamycin analogs (e.g., AP23573, Biolimus and ABT-578 (Abbott)), as well as those described in U.S. Patent Publication No. 2008/0207644 (Sonis et al.), U.S. Pat. No. 7,091,213 (Metcalf III et al.) (e.g., AP23573), WO 2004/026280, WO 2005/011688, WO 2005/070393, WO 2006/086172, U.S. Pat. Nos. 6,384,046, 6,197,781, 6,004,973, WO 2002/066019, 42-desmethoxy derivatives of rapamycin and its various analogs, as disclosed, e.g., in WO 2006/095185 (e.g., “39-desmethoxy”). All the references are incorporated herein by reference in their entireties.

In some embodiments, the composition may comprise one therapeutic agent. In one example, the composition may comprise an imidazotetrazine derivative compound. In one example, the composition may comprise temozolomide or an analog thereof. In one example, the composition may comprise temozolomide. In one example, the composition may comprise a taxane compound. In one example, the composition may comprise paclitaxel or an analog thereof. In one example, the composition may comprise paclitaxel. In one example, the composition may comprise rapamycin or an analog thereof. The composition may comprise rapamycin.

In some embodiments, a composition according to the present disclosure may comprise at least two (e.g., two, three, four, five, or more) therapeutic agents. At least two of the therapeutic agents may be hydrophobic. In some examples, all the therapeutic agents may be hydrophobic. In one example, the composition may comprise two therapeutic agents. In some examples, the composition may comprise an imidazotetrazine derivative compound and a taxane compound. In one example, the composition may comprise temozolomide or an analog thereof, and paclitaxel or an analog thereof. In one example, the composition may comprise temozolomide and paclitaxel. In some examples, the composition may comprise a taxane compound, and rapamycin or an analog thereof. In one example, the composition may comprise paclitaxel or an analog thereof, and rapamycin or an analog thereof. In one example, the composition may comprise paclitaxel and rapamycin. In some examples, the composition may comprise an imidazotetrazine derivative, and rapamycin or an analog thereof. In one example, the composition may comprise a temozolomide or an analog thereof, and rapamycin or an analog thereof. In one example, the composition may comprise temozolomide and rapamycin.

In some embodiments, the composition may carry a suitable amount of therapeutic agent(s). In some examples, the ratio of the total weight of the one or more therapeutic agents to the total weight of the first and the second polymers may be from 1% to 200%, e.g., from 1% to 90%, e.g., from 1% to 5%, from 5% to 10%, from 10% to 15%, from 15% to 20%, from 25% to 30%, from 35% to 40%, from 45% to 50%, from 55% to 60%, from 65% to 70%, from 70% to 75%, from 75% to 80%, from 80% to 85%, from 85% to 90%, from 90% to 95%, from 95% to 100%, from 100% to 105%, from 105% to 110%, from 110% to 115%, from 115% to 120%, from 120% to 125%, from 125% to 130% from 130% to 135%, from 135% to 140%, from 140% to 145%, from 145% to 150%, from 150% to 155%, from 155% to 160%, from 160% to 165%, from 165% to 170%, from 170% to 175%, from 175% to 180%, from 180% to 185%, from 185% to 190%, from 190% to 195%, from 195% to 200%, from 90% to 100%, from 95% to 105%, from 100% to 110%, from 105% to 115%, from 110% to 120%, from 115% to 125%, from 120% to 130%, from 125% to 135%, from 130% to 140%, from 135% to 145%, from 140% to 150%, from 25 to 70%, from 30% to 70%, from 40% to 70%, from 45% to 65%, from 45% to 60%, from 50% to 55%, from 55% to 65%, from 25% to 80%, from 30% to 75%, from 30% to 80%, from 30% to 85%, from 40% to 75%, or from 40% to 80%, from 40% to 85%, from 25% to 110%, from 30% to 110%, from 40% to 110%, from 30% to 100%, or from 40% to 100%.

In some examples, the ratio of the total weight of the one or more therapeutic agents to the total weight of the first and the second polymers is about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, about 111%, about 112%, about 113%, about 114%, about 125%, about 116%, about 117%, about 118%, about 119%, about 120%, about 121%, about 122%, about 123%, about 124%, about 125%, about 126%, about 127%, about 128%, about 129%, about 130%, about 131%, about 132%, about 133%, about 134%, about 135%, about 136%, about 137%, about 138%, about 139%, about 140%, about 141%, about 142%, about 143%, about 144%, about 145%, about 146%, about 147%, about 148%, about 149%, or about 150%.

In one example, the ratio of the total weight of the one or more therapeutic agents to the total weight of the first and the second polymers may be from 50% to 60%. In one example, the ratio of the total weight one or more therapeutic agents to the total weight of the first and the second polymers may be from 50% to 55%. In one example, the ratio of the total weight of the one or more therapeutic agents to the total weight of the first and the second polymers may be from 45% to 65%. In one example, the ratio of the total weight of the one or more therapeutic agents to the total weight of the first and the second polymers may be from 30% to 70%. In one example, the ratio of the total weight of the one or more therapeutic agents to the total weight of the first and the second polymers may be from 30% to 75%. In one example, the ratio of the total weight of the one or more therapeutic agents to the total weight of the first and the second polymers may be from 30% to 80%. In one example, the ratio of the total weight of the one or more therapeutic agents to the total weight of the first and the second polymers may be from 40% to 70%. In one example, the ratio of the total weight of the one or more therapeutic agents to the total weight of the first and the second polymers may be from 40% to 75%. In one example, the ratio of the total weight of the one or more therapeutic agents to the total weight of the first and the second polymers may be from 40% to 80%. In one example, the ratio of the total weight of the one or more therapeutic agents to the total weight of the first and the second polymers may be from 20% to 110%. In one example, the ratio of the total weight of the one or more therapeutic agents to the total weight of the first and the second polymers may be from 30% to 110%. In one example, the ratio of the total weight of the one or more therapeutic agents to the total weight of the first and the second polymers may be from 40% to 110%. In one example, the ratio of the total weight of the one or more therapeutic agents to the total weight of the first and the second polymers may be from 30% to 100%. In one example, the ratio of the total weight of the one or more therapeutic agents to the total weight of the first and the second polymers may be from 40% to 100%. In one example, the ratio of the total weight of the one or more therapeutic agents to the total weight of the first and the second polymers may be from 50% to 100%. In one example, the ratio of the total weight of the one or more therapeutic agents to the total weight of the first and the second polymers may be from 90% to 100%. In one example, the ratio of the total weight of the one or more therapeutic agents to the total weight of the first and the second polymers may be from 95% to 105%. In one example, the ratio of the total weight of the one or more therapeutic agents to the total weight of the first and the second polymers may be from 140% to 150%.

In some examples, the ratio of the total weight of the one or more therapeutic agents to the total weight of the first and the second polymers may be at least 1%, at least 2%, at least 3%, at least 5%, at least 10%, at least 20%, at least 30%, at least 40%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, or at least 200%.

The ratio of the total weight of the one or more therapeutic agents to the total weight of the first and the second polymers may be calculated as the total weight of the one or more therapeutic agents in the composition divided by the total weight of the first and the second polymers, and multiply by 100%. For example, in a formulation comprising 60 mg total weight of therapeutic agents and 60 mg total weigh of the first and the second polymers, the ratio of the weight of the one or more therapeutic agents to the total weight of the first and the second polymers 50%.

In some examples, the composition may comprise an temozolomide or an analog thereof (e.g., temozolomide) and the ratio of the weight of the imidazotetrazine derivative compound (e.g., temozolomide) to the total weight of the first and the second polymers may be from 25% to 80%, from 30% to 75%, from 30% to 70%, from 40% to 75%, from 25% to 35%, from 30% to 40%, from 35% to 45%, from 40% to 50%, from 45% to 55%, from 50% to 60%, from 55% to 65%, from 60% to 70%, from 65% to 75%, from 70% to 80%, or from 75% to 85% from 80% to 90%, from 85% to 95%, from 90% to 100%, from 95% to 100%, from 100% to 105%, from 105% to 110%, from 110% to 115%, or from 115% to 120%.

In some examples, the ratio of the weight of the temozolomide or an analog thereof (e.g., temozolomide) to the total weight of the first and the second polymers may be at least 20%, at least 25, at least 30%, at least 40%, at least 50%, at least 55%, at least 60%, at least 65%, at least 70%, at least 75%, at least 80%, at least 85%, at least 90%, at least 95%, or at least 100%.

In one example, the ratio of the weight of the temozolomide or an analog thereof (e.g., temozolomide) to the total weight of the first and the second polymers may be from 50% to 60%. In one example, the ratio of the weight of the temozolomide or an analog thereof (e.g., temozolomide) to the total weight of the first and the second polymers may be from 50% to 55%. In one example, the ratio of the weight of the temozolomide or an analog thereof (e.g., temozolomide) to the total weight of the first and the second polymers may be from 45% to 65%. In one example, the ratio of the weight of the temozolomide or an analog thereof (e.g., temozolomide) to the total weight of the first and the second polymers may be from 30% to 70%. In one example, the ratio of the weight of the temozolomide or an analog thereof (e.g., temozolomide) to the total weight of the first and the second polymers may be from 30% to 75%. In one example, the ratio of the weight of the temozolomide or an analog thereof (e.g., temozolomide) to the total weight of the first and the second polymers may be from 30% to 80%. In one example, the ratio of the weight of the temozolomide or an analog thereof (e.g., temozolomide) to the total weight of the first and the second polymers may be from 40% to 70%. In one example, the ratio of the weight of the temozolomide or an analog thereof (e.g., temozolomide) to the total weight of the first and the second polymers may be from 40% to 75%. In one example, the ratio of the weight of the temozolomide or an analog thereof (e.g., temozolomide) to the total weight of the first and the second polymers may be from 40% to 80%. In one example, the ratio of the weight of the temozolomide or an analog thereof (e.g., temozolomide) to the total weight of the first and the second polymers may be from 30% to 110%. In one example, the ratio of the weight of the temozolomide or an analog thereof (e.g., temozolomide) to the total weight of the first and the second polymers may be from 40% to 110%. In one example, the ratio of the weight of the temozolomide or an analog thereof (e.g., temozolomide) to the total weight of the first and the second polymers may be from 30% to 100%. In one example, the ratio of the weight of the temozolomide or an analog thereof (e.g., temozolomide) to the total weight of the first and the second polymers may be from 40% to 100%. In one example, the ratio of the weight of the temozolomide or an analog thereof (e.g., temozolomide) to the total weight of the first and the second polymers may be from 50% to 110%. In one example, the ratio of the weight of the temozolomide or an analog thereof (e.g., temozolomide) to the total weight of the first and the second polymers may be from 50% to 100%.

In some examples, the ratio of the weight of the temozolomide or an analog thereof (e.g., temozolomide) to the total weight of the first and the second polymers may be about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, or about 110%.

In some examples, the composition may comprise paclitaxel or an analog (e.g., paclitaxel), and the ratio of the weight of the paclitaxel or an analog (e.g., paclitaxel) to the total weight of the first and the second polymers may be from 1% to 10%, from 1.5% to 8%, from 1.5% to 6%, from 1.6% to 5%, from 1% to 20%, 1% to 5%, 5% to 10%, 10% to 15%, 15% to 20%, 20% to 25%, 25% to 30%, 30% to 35%, 35% to 40%, 40% to 45%, from 25% to 80%, from 30% to 75%, from 30% to 70%, from 40% to 75%, from 25% to 35%, from 30% to 40%, from 35% to 45%, from 40% to 50%, from 45% to 55%, from 50% to 60%, from 55% to 65%, from 60% to 70%, from 65% to 75%, from 70% to 80%, from 1.5% to 43%, from 1.5% to 45%, from 1% to 43%, or 1% from % to 45%.

In some examples, the ratio of the total weight of the paclitaxel or an analog (e.g., paclitaxel) to the total weight of the first and the second polymers may be at least at least 0.5%, at least 0.8%, at least 1%, at least 1.2%, at least 1.4%, at least 1.6%, at least 1.8%, at least 2%, at least 2.2%, at least 2.4%, at least 2.6%, at least 2.8%, at least 3%, at least 3.2%, at least 3.4%, at least 3.6%, at least 3.8%, at least 4%, at least 4.2%, at least 4.4%, at least 4.6%, at least 4.8%, at least 5%, at least 5.2%, at least 5.4%, at least 5.6%, at least 5.8%, at least 6%, at least 6.2%, at least 6.4%, at least 6.6%, at least 6.8%, at least 7%, at least 8%, at least 9%, at least 10%, at least 15%, at least 20%, at least 25, at least 30%, at least 40%, at least 50%, at least 55%, at least 60%, at least 65%, at least 70%, at least 75%, at least 80%, at least 85%, or at least 90%.

In one example, the ratio of the weight of the paclitaxel or an analog (e.g., paclitaxel) to the total weight of the first and the second polymers may be from 1% to 10%. In one example, the ratio of the weight of the paclitaxel or an analog (e.g., paclitaxel) to the total weight of the first and the second polymers may be from 1.5% to 8%. In one example, the ratio of the weight of the paclitaxel or an analog (e.g., paclitaxel) to the total weight of the first and the second polymers may be from 1.5% to 6%. In one example, the ratio of the weight of the paclitaxel or an analog (e.g., paclitaxel) to the total weight of the first and the second polymers may be from 1.6% to 5%. In one example, the ratio of the weight of the paclitaxel or an analog (e.g., paclitaxel) to the total weight of the first and the second polymers may be from 1% to 20%. In one example, the ratio of the weight of the paclitaxel or an analog (e.g., paclitaxel) to the total weight of the first and the second polymers may be from 1.5% to 43%. In one example, the ratio of the weight of the paclitaxel or an analog (e.g., paclitaxel) to the total weight of the first and the second polymers may be from 1.5% to 45%. In one example, the ratio of the weight of the paclitaxel or an analog (e.g., paclitaxel) to the total weight of the first and the second polymers may be from 20% to 50%. In one example, the ratio of the weight of the paclitaxel or an analog (e.g., paclitaxel) to the total weight of the first and the second polymers may be from 25% to 30%. In one example, the ratio of the weight of the paclitaxel or an analog (e.g., paclitaxel) to the total weight of the first and the second polymers may be from 30% to 35%. In one example, the ratio of the weight of the paclitaxel or an analog (e.g., paclitaxel) to the total weight of the first and the second polymers may be from 35% to 40%. In one example, the ratio of the weight of the paclitaxel or an analog (e.g., paclitaxel) to the total weight of the first and the second polymers may be from 40% to 45%. In one example, the ratio of the weight of the paclitaxel or an analog (e.g., paclitaxel) to the total weight of the first and the second polymers may be from 45% to 50%.

In some examples, the ratio of the weight of the paclitaxel or an analog (e.g., paclitaxel) to the total weight of the first and the second polymers may be about 1%, about 1.20%, about 1%, about 1.60%, about 2%, about 2.00%, about 2%, about 2.40%, about 3%, about 2.80%, about 3%, about 3.20%, about 3%, about 3.60%, about 4%, about 4.00%, about 4%, about 4.40%, about 5%, about 4.80%, about 5%, about 5.20%, about 5%, about 5.60%, about 6%, about 6.00%, about 6%, about 6.40%, about 7%, about 6.80%, about 7%, about 7.20%, about 7%, about 7.60%, about 8%, about 8.00%, about 8%, about 8.40%, about 9%, about 8.80%, about 9%, about 9.20%, about 9%, about 9.60%, about 10%, about 10.00%, about 10%, about 10.40%, about 11%, about 10.80%, about 11%, about 11.20%, about 11%, about 11.60%, about 12%, about 12.00%, about 12%, about 12.40%, about 13%, about 12.80%, about 13%, about 13.20%, about 13%, about 13.60%, about 14%, about 14.00%, about 14%, about 14.40%, about 15%, about 14.80%, about 15%, about 16%, about 17%, about 18%, about 19%, about 20%, about 21%, about 22%, about 23%, about 24%, about 25%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45%.

In some examples, the composition comprise a temozolomide or an analog thereof and a paclitaxel or an analog (e.g., temozolomide and paclitaxel), and the ratio of the total weight of the temozolomide or an analog thereof and the paclitaxel or an analog (e.g., temozolomide and paclitaxel) to the total weight of the first and the second polymers may be from 25% to 80%, from 30% to 75%, from 30% to 70%, from 40% to 75%, from 25% to 35%, from 30% to 40%, from 35% to 45%, from 40% to 50%, from 45% to 55%, from 50% to 60%, from 55% to 65%, from 60% to 70%, from 65% to 75%, from 70% to 80%, from 75% to 85%, from 80% to 90%, from 85% to 95%, from 90% to 100%, from 95% to 105%, from 100% to 110%, from 105% to 115%, from 110% to 120%, from 115% to 125%, from 120% to 130%, from 125% to 135%, from 130% to 140%, from 135% to 145%, or from 140% to 150%.

In some examples, the ratio of the total weight of the temozolomide or an analog thereof and the paclitaxel or an analog (e.g., temozolomide and paclitaxel) to the total weight of the first and the second polymers may be at least 20%, at least 25, at least 30%, at least 40%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 135%, at least 140%, at least 145%, or at least 150%.

In one example, the ratio of the total weight of the temozolomide or an analog thereof and the paclitaxel or an analog (e.g., temozolomide and paclitaxel) to the total weight of the first and the second polymers may be from 50% to 60%. In one example, the ratio of the total weight of the temozolomide or an analog thereof and the paclitaxel or an analog (e.g., temozolomide and paclitaxel) to the total weight of the first and the second polymers may be from 50% to 55%. In one example, the ratio of the total weight of the temozolomide or an analog thereof and the paclitaxel or an analog (e.g., temozolomide and paclitaxel) to the total weight of the first and the second polymers may be from 45% to 65%. In one example, the ratio of the total weight of the temozolomide or an analog thereof and the paclitaxel or an analog (e.g., temozolomide and paclitaxel) to the total weight of the first and the second polymers may be from 30% to 70%. In one example, the ratio of the total weight of the temozolomide or an analog thereof and the paclitaxel or an analog (e.g., temozolomide and paclitaxel) to the total weight of the first and the second polymers may be from 30% to 75%. In one example, the ratio of the total weight of the temozolomide or an analog thereof and the paclitaxel or an analog (e.g., temozolomide and paclitaxel) to the total weight of the first and the second polymers may be from 30% to 80%. In one example, the ratio of the total weight of the temozolomide or an analog thereof and the paclitaxel or an analog (e.g., temozolomide and paclitaxel) to the total weight of the first and the second polymers may be from 40% to 70%. In one example, the ratio of the total weight of the temozolomide or an analog thereof and the paclitaxel or an analog (e.g., temozolomide and paclitaxel) to the total weight of the first and the second polymers may be from 40% to 75%. In one example, the ratio of the total weight of the temozolomide or an analog thereof and the paclitaxel or an analog (e.g., temozolomide and paclitaxel) to the total weight of the first and the second polymers may be from 40% to 80%. In one example, the ratio of the total weight of the temozolomide or an analog thereof and the paclitaxel or an analog (e.g., temozolomide and paclitaxel) to the total weight of the first and the second polymers may be from 30% to 150%. In one example, the ratio of the total weight of the temozolomide or an analog thereof and the paclitaxel or an analog (e.g., temozolomide and paclitaxel) to the total weight of the first and the second polymers may be from 40% to 150%. In one example, the ratio of the total weight of the temozolomide or an analog thereof and the paclitaxel or an analog (e.g., temozolomide and paclitaxel) to the total weight of the first and the second polymers may be from 30% to 145%. In one example, the ratio of the total weight of the temozolomide or an analog thereof and the paclitaxel or an analog (e.g., temozolomide and paclitaxel) to the total weight of the first and the second polymers may be from 40% to 145%. In one example, the ratio of the total weight of the temozolomide or an analog thereof and the paclitaxel or an analog (e.g., temozolomide and paclitaxel) to the total weight of the first and the second polymers may be from 30% to 140%. In one example, the ratio of the total weight of the temozolomide or an analog thereof and the paclitaxel or an analog (e.g., temozolomide and paclitaxel) to the total weight of the first and the second polymers may be from 40% to 140%.

In one example, the ratio of the total weight of the temozolomide or an analog thereof and the paclitaxel or an analog (e.g., temozolomide and paclitaxel) to the total weight of the first and the second polymers may be about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 121%, about 122%, about 123%, about 124%, about 125%, about 126%, about 127%, about 128%, about 129%, about 130%, about 131%, about 132%, about 133%, about 134%, about 135%, about 136%, about 137%, about 138%, about 139%, about 140%, about 141%, about 142%, about 143%, about 144%, about 145%, about 146%, about 147%, about 148%, about 149%, or about 150%.

In some embodiments, the composition may comprise water. In some examples, the composition may be a hydrogel. In some examples, a hydrogel may be a thermosensitive hydrogel (i.e., may be in sol phase or gel phase depending on the temperature).

In some examples, the ratio of the weight of the water to the total weight of other components in the composition may be from 1:90 to 1:10, e.g., from 1:90 to 1:15, from 1:80 to 1:15, from 1:70 to 1:15, from 1:70 to 1:20, from 1:25 to 1:15, from 1:30 to 1:20, from 1:35 to 1:25, from 1:40 to 1:30, from 1:45 to 1:35, from 1:50 to 1:40, from 1:55 to 1:45, from 1:60 to 1:50, from 1:65 to 1:55, from 1:70 to 1:60, from 1:75 to 1:65, from 1:80 to 1:70, from 1:85 to 1:75, or from 1:90 to 1:80. In some examples, the ratio of the weight of the water to the total weight of other components in the composition may be from 1:80 to 1:15. In some examples, the ratio of the weight of the water to the total weight of other components in the composition may be from 1:70 to 1:20. In some examples, the ratio of the weight of the water to the total weight of other components in the composition may be about 1:90, about 1:85, about 1:80, about 1:75, about 1:70, about 1:65, about 1:60, about 1:55, about 1:50, about 1:45, about 1:40, about 1:35, about 1:30, about 1:25, about 1:20, about 1:15, about 1:10, or about 1:5.

In some examples, the ratio of the weight of the water to the total weight of other components in the composition may be from 90:1 to 10:1, e.g., from 90:1 to 15:1, from 80:1 to 15:1, from 70:1 to 15:1, from 70:1 to 20:1, from 25:1 to 15:1, from 30:1 to 20:1, from 35:1 to 25:1, from 40:1 to 30:1, from 45:1 to 35:1, from 50:1 to 40:1, from 55:1 to 45:1, from 60:1 to 50:1, from 65:1 to 55:1, from 70:1 to 60:1, from 75:1 to 65:1, from 80:1 to 70:1, from 85:1 to 75:1, or from 90:1 to 80:1. In some examples, the ratio of the weight of the water to the total weight of other components in the composition may be from 80:1 to 15:1. In some examples, the ratio of the weight of the water to the total weight of other components in the composition may be from 70:1 to 20:1. In some examples, the ratio of the weight of the water to the total weight of other components in the composition may be about 90:1, about 85:1, about 80:1, about 75:1, about 70:1, about 65:1, about 60:1, about 55:1, about 50:1, about 45:1, about 40:1, about 35:1, about 30:1, about 25:1, about 20:1, about 15:1, about 10:1, or about 5:1.

In some embodiments, the composition herein may be in a solid form (e.g., powder, tablet, wafer, or pellet). The solid form may become a hydrogel when mixed with water or an aqueous liquid (e.g., a solution or a bodily liquid). In some examples, for the mixture, the ratio of the weight of the solid form (in g) to the volume of the water (in mL) may be from 10% to 90%, e.g., from 15% to 90%, from 15% to 85%, from 15% to 80%, from 15% to 70%, from 20% to 70%, from 15% to 25%, from 20% to 30%, from 25% to 35%, from 30% to 40%, from 35% to 45%, from 40% to 50%, from 45% to 55%, from 50% to 60%, from 55% to 65%, from 60% to 70%, from 65% to 75%, from 70% to 80%, from 75% to 85%, or from 80% to 90% g/mL calculated as the total weight of the solid form by volume of water. In some examples, the ratio of the weight of the solid form (in g) to the volume of the water (in mL) may be from 20% g/mL to 70% g/mL calculated as the total weight of the solid form by volume of water. In some examples, the ratio of the weight of the solid form (in g) to the volume of the water (in mL) may be about 15%, about 20%, about 25%, about 30%, about 35%, about 40%, about 45%, about 50%, about 55%, about 60%, about 65%, about 70%, about 75%, about 80%, about 85%, or about 90% g/mL calculated as the total weight of the solid form by volume of water. The ratio of the weight of the solid form (in g) to the volume of the water (in mL) is calculated by dividing the weight of the solid form (in g) by the volume of the water (in mL), and multiply by 100%. For example, when a composition comprising 0.1 g solid form is mixed with 0.2 mL water, the ratio of the weight of the solid form (in g) to the volume of the water (in mL) is 50% g/mL.

### Additional Components

The compositions may comprise one or more additional components besides the first and the second polymers, therapeutic agent(s), and water.

In some embodiments, the composition may comprise one or more bulking agents. A bulking agent may be a compound that may increase the strength of the gel when the composition is in a gel phase, increase the final volume or weight of the composition (e.g., for easy formulation or administration), add bulk to the composition, and/or assist in the control of the properties of the composition during lyophilization. Examples of bulking agents include mannitol, lactose sucrose, dextran, trehalose, glycine, dextran, polyvinylpyrrolidone, inositol, sorbitol, dimethylsulfoxide, glycerol, and albumin. In one example, the bulking agent may be mannitol.

In some examples, the ratio of the weight of the bulking agent(s) to the weight of the first and the second polymers in the composition may be from 1:20 to 1:1, from 1:10 to 1:2, from 1:9 to 1:3, from 1:8 to 1:4, from 1:7 to 1:5, from 1:20 to 1:16, from 1:18 to 1:14, from 1:16 to 1:12, from 1:14 to 1:10, from 1:12 to 1:8, from 1:10 to 1:6, from 1:8 to 1:4, or from 1:6 to 1:2. In some examples, the ratio of the weight of the bulking agent(s) to the weight of the first and the second polymers in the composition may be from 1:5 to 1:4. In some examples, the ratio of the weight of the bulking agent(s) to the weight of the first and the second polymers in the composition may be about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4, about 1:3, about 1:2, or about 1:1. In one example, the ratio of the weight of the bulking agent(s) to the weight of the first and the second polymers in the composition may be about 1:6. The ratio of the weight of the bulking agent(s) to the weight of the first and the second polymers is calculated as (the total weight of the bulking agent(s)): (the total weight of the first and second polymers). For example, in a composition comprising 10 mg bulking agent and 60 mg total weight of PLGA-PEG-PLGA polymers, the ratio between the weight of the bulking agent(s) and the weight of the polymers is 1:6.

In some embodiments, the composition may consist of the first and the second polymers and the one or more therapeutic agents. In some embodiments, the composition may consist of the first and the second polymers, the one or more therapeutic agents, and water. In some embodiments, the composition may consist of the first and the second polymers, the one or more therapeutic agents, water, and one or more bulking agents.

In some embodiments, the composition may further comprise an ointment base. In some examples, such composition may be administered topically. Examples of ointment base include anhydrous absorption base (e.g., Hydrophilic petrolatum, Lanolin, Aquaphor, Aquabase, Polysorb), water-in-oil emulsion base (e.g., Hydrous lanolin, Cold cream, Eucerin, Hydrocream, Nivea), and oil-in-water emulsion base (e.g., Hydrophilic ointment, vanishing cream, Dermabase, Velvachol). In one example, the ointment base may be Aquaphor.

In some examples, the ratio between the weight of the drug and the volume of the ointment may be at least 1, at least 5, at least 10, at least 15, at least 20, at least 25, at least 30, at least 35, at least 40, at least 45, or at least 50 mg drug/1 mL ointment. In some examples, the ratio between the weight of the drug and the volume of the ointment may be from 10 to 50, from 15 to 35, from 20 to 35, from 1 to 5, from 2.5, to 7.5, from 5 to 10, from 7.5 to 12.5, from 10 to 15, from 12.5 to 17.5, from 15 to 20, from 17.5 to 22.5, from 20 to 25, from 22.5 to 27.5, from 25 to 30, from 27.5 to 32.5, from 30 to 35, from 32.5 to 37.5, or from 35 to 40 mg drug/1 mL ointment.

In some embodiments, the composition may comprise a mixture of sol-gel formulation and an ointment base. For example, the ratio between the volume of the sol-gel formulation and the volume of the ointment base may be from 1:1 to 1:50, e.g., 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, or 1:50.

### Formulations

The composition may be in a desired form of formulation. In some embodiments, the composition may be in the form a hydrogel. In some embodiments, the composition may be in the form a thermosensitive hydrogel (in the sol or gel phase depending on the temperature).

In some embodiments, the composition may be in a solid form. The solid form may be reconstituted into a hydrogel (e.g., thermosensitive hydrogel (in the sol or gel phase depending on the temperature)) by being mixed with water or an aqueous liquid (e.g., a solution or a bodily fluid). For example, the composition may be in the form of powders. In some examples, the composition may be in the form of pellets, e.g., made from pressing powders in to a pellet shape. In some examples, each of the pellets may have a weight from 5 mg to 30 mg, from 10 mg to 25 mg, from 10 mg to 24 mg, from 10 mg to 23 mg, from 10 mg to 22 mg, from 10 mg to 21 mg, from 11 mg to 25 mg, from 11 mg to 24 mg, from 11 mg to 23 mg, from 11 mg to 22 mg, from 11 mg to 21 mg, from 12 mg to 25 mg, from 12 mg to 24 mg, from 12 mg to 23 mg, from 12 mg to 22 mg, from 12 mg to 21 mg, from 13 mg to 20 mg, or from 14 mg to 19 mg. In some examples, each of the pellets may have a weight of about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, or about 30 mg or more.

For treatment, one or more pellets may be administered to the subject. For example, 1, 2, 3, 4, 5, 6, 7, 8, 9, 10, 11, 12, 13, 14, 15, 16, 17, 18, 19, 20 or more pellets may be administered to the subject.

### Kits and Devices

The present disclosure also provides kits and devices comprising the composition herein. In some embodiments, the device may be an injection device (e.g., a syringe) pre-filled with the composition (e.g., in a hydrogel form (either sol or gel phase) or a solid form (can be reconstituted into a hydrogel with water).

### Method of Making

The present disclosure further provides methods of making the compositions herein. In general, the methods may comprise (a) mixing a first polymer, a second polymer, and one or more therapeutic agents in water; (b) mixing an organic solvent with the mixture from a; and (c) lyophilizing the mixture from b. In some examples, (a) may comprise mixing a solution of the first polymer, a solution of the second polymer, and the one or more therapeutic agents. In some examples, (a) may comprise mixing a first polymer, a second polymer, and a first therapeutic agent in water, and (b) comprises mixing a second therapeutic agent with the organic solvent with the mixture of (a).

In some examples, (a) may comprise mixing a first polymer, a second polymer, a bulking agent. and a first therapeutic agent in water, and (b) comprises mixing a second therapeutic agent with the organic solvent with the mixture of (a).

Examples of the organic solvent include C1-C3 alcohols such as ethanol, 1,2-propylene glycol, glycerol and isopropanol, 1,4-butane diol, 1,6 hexane diol, n-methylpyrrolidinone, dimethyl sulfoxide, ethyl lactate, acetone, methyl ethyl ketone, polyethylene glycol and its derivatives. In one example, the organic solvent may be acetone.

In some embodiments, the method of making the composition may further comprise freeze-drying (e.g., at about −80° C.) the mixture of (b) prior to (c).

In some embodiments, the polyester block of the PLGA-PEG-PLGA polymer can be synthesized from a variety of monomers such as D,L-lactide, D-lactide, L-lactide, D,L-lactic acid, D-lactic acid, L-lactic acid, glycolide, glycolic acid, F-caprolactone, 6-hydroxyhexanoic acid, γ-butyrolactone, 4-hydroxybutyric acid, 6-valerolactone, 5-hydroxyvaleric acid, hydroxybutyric acids, malic acid, and copolymers thereof. For purpose of illustration, the A-block copolymers are generally lactide or lactide-co-glycolide moieties. However, unless specifically referred to otherwise, the terms “lactide”, “lactate”, or “L” include all lactic acid derivatives and “glycolide”, “glycolate”, or “G” include all glycolic acid derivatives. In the hydrophobic polyester A-block, the molar ratio of lactate content to glycolate content (L:G ratio) can be from 3:1 to 1:3, 2:1 to 1:2, 1.5:1 to 1:1.5. The L:G ratio may be 0.8, 0.9, 1, 1.1, or 1.2. In an example, the L:G ratio may be about 1.

In some embodiments, two or more different PLGA-PEG-PLGA polymers can be used to prepare a thermogel composition, for example, the combinations described in U.S. Pat. No. 7,135,190 (Piao et al.). Mixing of two or more types of ABA or BAB triblock polyester polyethylene glycol copolymers can be done by mixing two or more individually synthesized triblock copolymers, or by synthesizing two or more tri-block copolymers in one reaction vessel. The mixture of copolymers resulting from these processes can have the same or different gelation properties. For example, a dual polymer system can be prepared with both polymers having polyester A blocks with the same lactide/glycolide ratio, molecular weight and polydispersity, and different B (PEG) block molecular weights.

The triblock PLGA-PEG-PLGA copolymer may be synthesized by ring opening polymerization or condensation polymerization, for example, as described by U.S. Pat. No. 6,004,573 (Rathi et al.) and U.S. Pat. No. 7,135,190 (Piao et al.), which are incorporated by reference herein in their entireties.

### Therapeutic Applications

Further provided herein are methods of treating diseases or other health conditions using the compositions herein. In general, the methods comprise administering the composition to a subject in need thereof.

In some embodiments, the composition according to the present disclosure may be administered locally. Compared to systemic administration, local administration may be used to deliver the therapeutic agent(s) to its target location at a higher dose thus having lower toxicity to other tissue or organs. In some examples, the amount of the therapeutic agent(s) administered to the subject would cause systemic toxicity if administered orally or intravenously, while the local administration causes lower or no systemic toxicity.

The methods may also allow delivering the therapeutic agent(s) that cannot readily reach (e.g., cannot reach or reach with only low dose) its target location if administered systemically. For example, the methods may be used to treat diseases in the brain by intracranial administration (e.g., implantation) of the composition so that the therapeutic agent(s) may be delivered directly to the brain, bypassing the blood brain barrier. The methods may be used to deliver therapeutic agent(s) that would be blocked by the blood brain barrier if administered systemically and/or to deliver the therapeutic agent(s) at a high local dose that cannot be reached by systemic administration.

In some embodiments, the composition may be administered to (or a location in proximity to) the target tissue or organ of the therapeutic agent(s). In some examples, when the disease is cancer, the composition may be administered to the tumor tissue, to the tissue or organ where the tumor tissue is located, or to a location in proximity to the tumor tissue so that, once released from the composition, the therapeutic agent(s) can reach the tumor tissue/cells.

In some examples, the composition may be administered after the surgical removal of the tumor or a portion thereof. In some examples, the composition may be administered to the surgical resection cavity. In such cases, the composition may treat the remaining portion of the tumor and/or prevent reoccurrence. In some examples, the composition may be administered without surgery. In such cases, the composition may be used to treat tumors that are not suitable for surgery (e.g., due to the patient's intolerance) or cannot be surgically removed (e.g., due to the location, size, etc.).

In some embodiments, the method may be used to treat a cancer. In some examples, the cancer may be brain cancer, e.g., gliomas (e.g., astrocytoma, oligodendroglial tumor, and glioblastoma), acoustic neuroma, brain metastases, choroid plexus carcinoma, craniopharyngioma, embryonal tumors, ependymoma, medulloblastoma, meningioma, pediatric brain tumors, pineoblastoma, or pituitary tumors. In some examples, the cancer may be glioblastoma (e.g., newly diagnosed, recurrent, relapsed, or progressive). In some examples, the cancer may be astrocytoma (e.g., anaplastic astrocytoma). In some examples, the cancer may be melanoma. In some examples, the cancer may be anaplastic gliomas, central neural system (CNS) metastases (e.g., from solid tumors), Cutaneous T-cell lymphomas (e.g., mycosis fungoides and Sezary syndrome), Ewing sarcoma, pancreatic neuroendocrine tumors, lymphoma (e.g., CNS lymphoma), soft tissue sarcomas, hemangiopericytoma, or solitary fibrous tumor.

In some examples, the cancer may be Kaposi sarcoma, breast cancer, lung cancer (e.g., non-small cell lung cancer), ovarian cancer, or adenocarcinoma (e.g., adenocarcinoma of the pancreas). In some examples, the cancer may be angiosarcoma, bladder cancer, esophagus cancer, prostate cancer, cervical cancer, gastric cancer, head and neck cancer, lymphoma, neoplasm of endometrium of corpus uteri, nasopharynx cancer, myeloma, small cell lung cancer, testicular cancer, anal cancer, endometrial cancer, esophageal cancer, melanoma, penile, soft tissue sarcoma, testicular germ cell tumors, thymoma/thymic carcinoma, thyroid cancer, or unknown primary adenocarcinoma.

Additional examples of cancers include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocelluar carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

Examples of disease also include restenosis of blood vessels (e.g., coronary or peripheral blood vessels).

In some embodiments, the composition may be administered in the sol phase. After the solution is administered, it may transition to the gel phase (e.g., around body temperature). Administering the composition in the sol phase may allow easy handling and administration. For example, when injected to a cavity in a subject, the solution may be able to fill the cavity. So when the solution forms a gel, the gel can conform with the shape and/or size of the cavity very well. This can facilitate optimal drug release and penetration profiles.

In some embodiments, the composition may be administered as a solid form. The solid form may be powder or pellets as described herein. After administration, the solid form may absorb liquid around and becomes a solution or a gel. In some cases, this approach allows easy administration provided by the solid form and also takes advantage of the flexibility of a thermosensitive hydrogel. In some embodiments, the solid form may have longer shelf-life and allow easy and low-cost shipping and storing.

In some embodiments, the composition may be administered in the gel phase. The gelation may be the result of raising the temperature of a drug laden polymer solution above the gelation temperature of the polymer prior to administration or caused by raising the concentration of the polymer in the solution above the saturation concentration at the temperature of administration.

The composition may be administered intracranially, parenterally, topically, transdermally or inserted into a cavity such as by intracranial, ocular, vaginal, transurethral, rectal, nasal, oral, or aural administration. In some embodiments, the composition may be administered intracranially. In some embodiments, the composition may be administered parenterally. In some embodiments, the composition may be administered topically. In some embodiments, the composition may be administered intranasally.

### Combination Therapies

The methods of treatment may comprise administering the composition herein and one or more additional therapeutic agents. The additional therapeutic agent(s) may be administered before, after, or simultaneously with the administration of the composition herein. In some examples, the additional therapeutic agent(s) may be administered systemically. In some examples, the additional therapeutic agent(s) may be administered locally.

The additional therapeutic agents may include chemotherapeutic agents. Examples of chemotherapeutic agents include aminoglutethimide, amsacrine, asparaginase, bcg, anastrozole, bleomycin, buserelin, bicalutamide, busulfan, capecitabine, carboplatin, camptothecin, chlorambucil, cisplatin, carmustine, cladribine, colchicine, cyclophosphamide, cytarabine, dacarbazine, cyproterone, clodronate, daunorubicin, diethylstilbestrol, docetaxel, dactinomycin, doxorubicin, dienestrol, etoposide, exemestane, filgrastim, fluorouracil, fludarabine, fludrocortisone, epirubicin, estradiol, gemcitabine, genistein, estramustine, fluoxymesterone, flutamide, goserelin, leuprolide, hydroxyurea, idarubicin, levamisole, imatinib, lomustine, ifosfamide, megestrol, melphalan, interferon, irinotecan, letrozole, leucovorin, ironotecan, mitoxantrone, nilutamide, medroxyprogesterone, mechlorethamine, mercaptopurine, mitotane, nocodazole, octreotide, methotrexate, mitomycin, paclitaxel, oxaliplatin, temozolomide, pentostatin, plicamycin, suramin, tamoxifen, porfimer, mesna, pamidronate, streptozocin, teniposide, procarbazine, titanocene dichloride, raltitrexed, testosterone, thioguanine, vincristine, vindesine, thiotepa, topotecan, tretinoin, vinblastine, and vinorelbine. The additional therapeutic agents may be biologics, e.g., rituximab or trastuzumab, an immune therapy agent such as anti-PD-1 antibody, anti-PD-L1 antibody, and/or anti-TIGIT antibody.

The present application also provides aspects and embodiments as set forth in the following numbered Statements:

Statement 1. A composition comprising: a first polymer and a second polymer; and one or more therapeutic agents, wherein: (i) the first and the second polymers are PLGA-PEG-PLGA triblock copolymers, (ii) the first polymer has a molecular weight higher than the second polymer, (iii) a molar ratio of the first polymer to the second polymer is from 3:1 to 1:1, and (iv) a ratio of a total weight of the one or more therapeutic agents to a total weight of the first and second polymers is at least 2% (e.g., at least 30%).

Statement 2. The composition of Statement 1, wherein the ratio of the total weight of the one or more therapeutic agents to the total weight of the first and second polymers is from 30% to 80%.

Statement 3. The composition of Statement 1, wherein the ratio of the total weight of the one or more therapeutic agents to the total weight of the first and second polymers is from 45% to 65%.

Statement 4. The composition of any one or combination of the Statements 1-3, wherein the ratio of the weight of the one or more therapeutic agents to the total weight of the first and the second polymers is from 58% to 62%.

Statement 5. The composition of any one or combination of the Statements 1-3, wherein the ratio of the weight of the one or more therapeutic agents to the total weight of the first and the second polymers is at least 90%.

Statement 6. The composition of any one or combination of the Statements 1-3, wherein the ratio of the weight of the one or more therapeutic agents to the total weight of the first and the second polymers is from 30% to 145%.

Statement 7. The composition of any one or combination of the Statements above, further comprising water, wherein a ratio of a weight of the water to a total weight of other components in the composition is from 80:1 to 15:1.

Statement 8. The composition of Statement 7, wherein the ratio of a weight of the water to a total weight of other components in the composition is 70:1 to 20:1.

Statement 9. The composition of any one or combination of the Statements above, wherein the molar ratio between the first and the second polymers is about 2:1.

Statement 10. The composition of any one or combination of Statements 1-9, wherein the composition is a hydrogel.

Statement 11. The composition of Statement 10, wherein the composition is a non-flowing thermosensitive hydrogel at a temperature from 28° C. to 50° C. and a free-flowing solution at a temperature from 0° C. to 26° C.

Statement 12. The composition of any one or combination of the Statements above, wherein the composition is in a solid form, and the solid form becomes a hydrogel when mixing with water or an aqueous solution from about 20% g/mL to 70% g/mL measured as total weight of the solid form by volume of water.

Statement 13. The composition of any one or combination of the Statements above, further comprising a bulking agent.

Statement 14. The composition of Statement 13, wherein a ratio of the total weight of the first polymer, the second polymer and the therapeutic agents to a weight of the bulking agent is at least 9.

Statement 15. The composition of Statement 13 or 14, wherein the bulking agent is mannitol.

Statement 16. The composition of any one or combination of the Statements above, wherein the one or more therapeutic agents comprises one or more chemotherapeutic drugs.

Statement 17. The composition of Statement 16, wherein the one or more chemotherapeutic drugs are hydrophobic.

Statement 18. The composition of Statement 16, wherein the one or more chemotherapeutic drugs comprises a taxane compound.

Statement 19. The composition of Statement 18, wherein the taxane compound is paclitaxel or an analog thereof.

Statement 20. The composition of Statement 19, wherein a ratio of a weight of the paclitaxel or an analog thereof to the total weight of the first and the second polymers is from 1.5% to 5%.

Statement 21. The composition of Statement 19, wherein a ratio of a weight of the paclitaxel or an analog thereof to the total weight of the first and the second polymers is from 1.5% to 43%.

Statement 22. The composition of Statement 16, wherein the one or more chemotherapeutic drugs comprises an imidazotetrazine derivative compound.

Statement 23. The composition of Statement 22, wherein the imidazotetrazine derivative compound is temozolomide or an analog thereof.

Statement 24. The composition of claim 23, wherein a ratio of a weight of the temozolomide or an analog thereof to the total weight of the first and the second polymers is from 30% to 70%.

Statement 25. The composition of claim 23, wherein a ratio of a weight of the temozolomide or an analog thereof to the total weight of the first and the second polymers is from 30% to 100%.

Statement 26. The composition of any one of the Statements above, which comprises two therapeutic agents.

Statement 27. The composition of Statement 26, comprising two chemotherapeutic drugs.

Statement 28. The composition of Statement 27, wherein the two chemotherapeutic drugs comprise an imidazotetrazine derivative compound and a taxane compound.

Statement 29. The composition of Statement 28, wherein the one or more chemotherapeutic drugs comprises paclitaxel or an analog thereof, and temozolomide or an analog thereof.

Statement 30. The composition of Statement 29, wherein (i) a ratio of the total weight of a weight of the paclitaxel or analog thereof to the total weight of the first and the second polymers is from 1.5% to 5%, and (ii) a ratio of a weight of the temozolomide or analog thereof to the total weight of the first and the second polymers is 30% to 70%.

Statement 31. The composition of Statement 29, wherein (i) a ratio of the total weight of a weight of the paclitaxel or analog thereof to the total weight of the first and the second polymers is from 1.5% to 43%, and (ii) a ratio of a weight of the temozolomide or analog thereof to the total weight of the first and the second polymers is 30% to 100%.

Statement 32. The composition of any one or combination of Statements above, which is in a form of powder.

Statement 33. The composition of any one or combination of Statements above, which is in a form of a pellet.

Statement 34. The composition of Statement 33, wherein the pellet has a weight from 3 mg to 10 mg.

Statement 35. The composition of Statement 33, wherein the pellet has a weight from 5 mg to 7 mg.

Statement 36. The composition of any or combination of Statements above, further comprising an ointment base.

Statement 37. A method of treating a disease, the method comprising administering the composition of any one or combination of the Statements above to a subject in need thereof.

Statement 38. The method of Statement 37, wherein the disease is cancer.

Statement 39. The method of Statement 38, wherein the cancer is glioblastoma or astrocytoma.

Statement 40. The method of Statement 38, wherein the cancer is melanoma.

Statement 41. The method of Statement 38, wherein the cancer is anaplastic gliomas, central neural system (CNS) metastases, Cutaneous T-cell lymphomas, Ewing sarcoma, pancreatic neuroendocrine tumors, lymphoma, soft tissue sarcomas, hemangiopericytoma, or solitary fibrous tumor.

Statement 42. The method of Statement 38, wherein the cancer is Kaposi sarcoma, breast cancer, lung cancer, ovarian cancer, or adenocarcinoma.

Statement 43. The method of Statement 38, wherein the cancer is angiosarcoma, bladder cancer, esophagus cancer, prostate cancer, cervical cancer, gastric cancer, head and neck cancer, lymphoma, neoplasm of endometrium of corpus uteri, nasopharynx cancer, myeloma, small cell lung cancer, testicular cancer, anal cancer, endometrial cancer, esophageal cancer, melanoma, penile, soft tissue sarcoma, testicular germ cell tumors, thymoma/thymic carcinoma, thyroid cancer, or unknown primary adenocarcinoma.

Statement 44. The method of any one or combination of Statements 37-43, wherein the composition is administered locally.

Statement 45. The method of Statement 44, wherein the composition is administered intracranially.

Statement 46. The method of claim 45, wherein the composition is administered topically.

Statement 47. The method of claim 46, wherein the amount of one or more therapeutic agents administered to the subject would cause systemic toxicity if administered systemically, and wherein the administration causes decreased or no systemic toxicity.

Statement 48. A method of producing a composition comprising: (a) mixing a first polymer, a second polymer, and one or more therapeutic agents in water; (b) mixing an organic solvent with the mixture from (a); and (c) lyophilizing the mixture from (b), wherein (i) the first and the second polymers are PLGA-PEG-PLGA triblock copolymers, (ii) the first polymer has a molecular weight higher than the second polymer, (iii) a molar ratio of the first polymer to the second polymer is from 3:1 to 1:1, and (iv) a ratio of a weight of the one or more therapeutic agents to a total weight of the first and second polymers is at least 30%.

Statement 49. The method of Statement 48, wherein (a) comprises mixing a solution of the first polymer, a solution of the second polymer, and the one or more therapeutic agents.

Statement 50. The method of Statement 48 or 49, wherein the one or more therapeutic agent is temozolomide.

Statement 51. The method of any one or combination of Statements 48-50, wherein (a) comprises mixing the first polymer, the second polymer, and a first therapeutic agent in water, and (b) comprises mixing a second therapeutic agent with the organic solvent with the mixture of (a).

Statement 52. The method of any one or combination of Statements 48-51, wherein (a) comprises mixing the first polymer, the second polymer, a bulking agent. and a first therapeutic agent in water, and (b) comprises mixing a second therapeutic agent with the organic solvent with the mixture of (a).

Statement 53. The method of Statement 52, wherein the bulking agent is mannitol.

Statement 54. The method of any one or combination of Statements 48-53, wherein the first therapeutic agent is temozolomide and the second therapeutic agent is paclitaxel.

Statement 55. The method of any one or combination of Statements 48-54, wherein the organic solvent is acetone.

Statement 56. The method of any one or combination of Statements 48-55, further comprising freeze-drying the mixture of (b) prior to (c).

Statement 57. Use of the composition of any one or combination of Statements 1-36 for the manufacture of a medicament for treating a disease.

Statement 58. The use of Statement 57, wherein the disease is cancer.

Statement 59. The use of Statement 58, wherein the cancer is glioblastoma or astrocytoma.

Statement 60. The use of Statement 58, wherein the cancer is melanoma.

Statement 61. The use of Statement 58, wherein the cancer is anaplastic gliomas, central neural system (CNS) metastases, Cutaneous T-cell lymphomas, Ewing sarcoma, pancreatic neuroendocrine tumors, lymphoma, soft tissue sarcomas, hemangiopericytoma, or solitary fibrous tumor.

Statement 62. The use of Statement 58, wherein the cancer is Kaposi sarcoma, breast cancer, lung cancer, ovarian cancer, or adenocarcinoma.

Statement 63. The use of Statement 58, wherein the cancer is angiosarcoma, bladder cancer, esophagus cancer, prostate cancer, cervical cancer, gastric cancer, head and neck cancer, lymphoma, neoplasm of endometrium of corpus uteri, nasopharynx cancer, myeloma, small cell lung cancer, testicular cancer, anal cancer, endometrial cancer, esophageal cancer, melanoma, penile, soft tissue sarcoma, testicular germ cell tumors, thymoma/thymic carcinoma, thyroid cancer, or unknown primary adenocarcinoma.

A kit comprising the composition of any one or combination of Statements 1-36.

### EXAMPLES

### Example 1

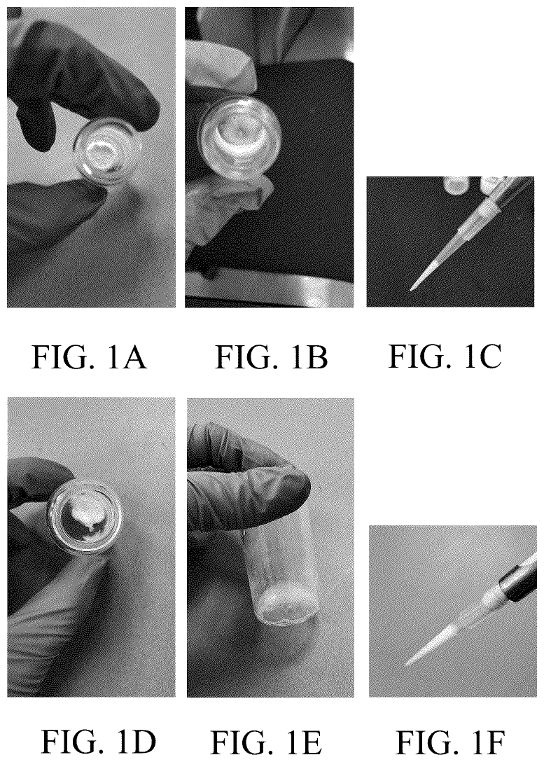

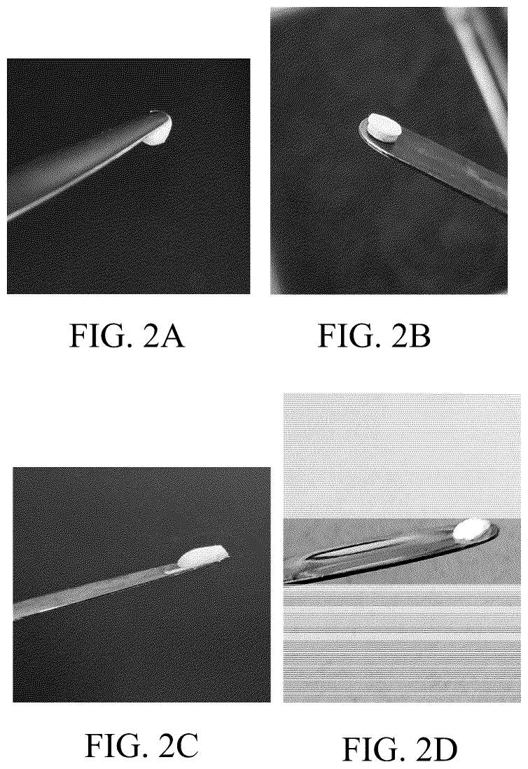

This example shows exemplary methods of preparing and characterizing thermosensitive hydrogels and solid pellets that carries temozolomide (TMZ), paclitaxel, or both TMZ and paclitaxel. In general, the thermosensitive gels produced in the examples were in a free-flowing sol phase below 30° C. (FIGS. 1B, 1C, 1E, and 1F) and was in a gel phase at 35° C. to 37° C. (FIGS. 2A and 2C).

Preparation of Thermosensitive Hydrogels and Solid Pellets Vehicle without Therapeutic Agents

PLGA-PEG-PLGA (1.5k-1.5k-1.5k Mw) triblock copolymer was dissolved in water at 200 mg/mL at 20-22° C. PLGA-PEG-PLGA (1k-1k-1k Mw) triblock copolymer was dissolved in water at 200 mg/mL at or below 10° C. In a sterilized glass scintillation vial, 200 mcL of PLGA-PEG-PLGA (1.5k-1.5k-1.5k) (200 mg/mL), 100 mcL of PLGA-PEG-PLGA (1k-1k-1k) (200 mg/mL), and 10 mg mannitol were mixed with 2.7 mL of distilled water. 1-3 mL of acetone was added to the mixture and vortexed vigorously for 1-2 min at 20-22° C. The final mixture was filtered through 0.2 micrometer regenerated cellulose (RC) filter and then frozen at −80° C. for at least 30 min. The frozen mixture was then lyophilized (below 0.8 mbar at −45° C.) for 24 h. The lyophilized cake was then rehydrated with 200 mcL of sterilized distilled water at 20-22° C. Rehydration time was 10-60 min, depending on the formulation. For pellets, the lyophilized cake was divided into five units and hand-pressed using sterilized hand pellet pressor into pellets at 3-mm diameter and 1 mm thickness, each weighing 4-9 mg. PLGA-PEG-PLGA (1.5k-1.5k-1.5k Mw) triblock copolymer alone does not form a gel at 37° C. (body temperature), remaining as a free-flowing sol (FIG. 4A). PLGA-PEG-PLGA (1k-1k-1k Mw) triblock copolymer alone forms a gel at 20-22° C. (ambient temperature) (FIG. 4B).

### Preparation of Thermosensitive Hydrogels and Solid Pellets Carrying TMZ

PLGA-PEG-PLGA (1.5k-1.5k-1.5k Mw) triblock copolymer was dissolved in water at 200 mg/mL at 20-22° C. PLGA-PEG-PLGA (1k-1k-1k Mw) triblock copolymer was dissolved in water at 200 mg/mL at or below 10° C. In a sterilized glass scintillation vial, 200 mcL of PLGA-PEG-PLGA (1.5k-1.5k-1.5k) (200 mg/mL), 100 mcL of PLGA-PEG-PLGA (1k-1k-1k) (200 mg/mL), 10 mg mannitol, and 30 mg TMZ were mixed with 3.7 mL of distilled water. 1 mL of acetone at was added to the mixture and vortexed vigorously for 1-2 min at 20-22° C. The final mixture was then filtered through 0.2 micrometer regenerated cellulose (RC) filter and frozen at −80° C. for at least 30 min. The frozen mixture was then lyophilized (below 0.8 mbar at −45° C.) for 24 h (FIG. 1D). The lyophilized cake was then rehydrated with 200 mcL of sterilized distilled water at 20-22° C. (FIGS. 1E and 1F). Rehydration time was 10-60 min, depending on the formulation. For pellets, the lyophilized cake was divided into five units and hand-pressed using sterilized hand pellet pressor into pellets at 3-mm diameter and 1 mm thickness, each weighing 4-9 mg (FIG. 2D).

### Preparation of Thermosensitive Hydrogels and Solid Pellets Carrying Paclitaxel

PLGA-PEG-PLGA (1.5k-1.5k-1.5k Mw) triblock copolymer was dissolved in water at 200 mg/mL at 20-22° C. PLGA-PEG-PLGA (i1k-1k-1k Mw) triblock copolymer was dissolved in water at 200 mg/mL at or below 10° C. In a sterilized glass scintillation vial, 200 mcL of PLGA-PEG-PLGA (1.5k-1.5k-1.5k) (200 mg/mL), 100 mcL of PLGA-PEG-PLGA (i1k-1k-1k) (200 mg/mL), and 10 mg mannitol were mixed with 2.7 mL of distilled water. Paclitaxel dissolved in acetone at 2 mg/1 mL, and 1 mL of the acetone with 2 mg/mL paclitaxel was added to the mixture and vortexed vigorously for 1-2 min at 20-22° C. The final mixture was then filtered through 0.2 micrometer regenerated cellulose (RC) filter and frozen at −80° C. for at least 30 min. The frozen mixture was then lyophilized (below 0.8 mbar at −45° C.) for 24 h. The lyophilized cake was then rehydrated with 200 mcL of sterilized distilled water at 20-22° C. Rehydration time is 10-60 min, depending on the formulation. For pellets, the lyophilized cake was divided into five units and hand-pressed using sterilized hand pellet pressor into pellets at 3-mm diameter and 1 mm thickness, each weighing 4-9 mg.

### Preparation of Thermosensitive Hydrogels and Solid Pellets Carrying Both TMZ and Paclitaxel

PLGA-PEG-PLGA (1.5k-1.5k-1.5k Mw) triblock copolymer was dissolved in water at 200 mg/mL at 20-22° C. PLGA-PEG-PLGA (1k-1k-1k Mw) triblock copolymer was dissolved in water at 200 mg/mL at or below 10° C. In a sterilized glass scintillation vial, 200 mcL of PLGA-PEG-PLGA (1.5k-1.5k-1.5k) (200 mg/mL), 100 mcL of PLGA-PEG-PLGA (1k-1k-1k) (200 mg/mL), 10 mg mannitol, and 30 mg TMZ were mixed with 3.7 mL of distilled water. Paclitaxel dissolved in acetone at 2 mg/1 mL, and 1 mL of the acetone with 2 mg/mL paclitaxel was added to the mixture and vortexed vigorously for 1-2 min at 20-22° C. The final mixture was then filtered through 0.2 micrometer regenerated cellulose (RC) filter and frozen at −80° C. for at least 30 min. The frozen mixture was then lyophilized (below 0.8 mbar at −45° C.) for 24 h. The lyophilized cake, as shown in FIG. 1A, was then rehydrated with 200 mcL of sterilized distilled water at 20-22° C. (FIGS. 1B and 1C). Rehydration time is 10-60 min, depending on the formulation. For pellets, the lyophilized cake was divided into five units and hand-pressed using sterilized hand pellet pressor into pellets at 3 mm diameter and 1 mm thickness, each weighing 4-9 mg (FIG. 2B). In some embodiments, when prepare the formulations, 1.5 mg to 11.2 mg paclitaxel may be dissolved in 1 mL acetone to make formulations with different doses of paclitaxel.

### Quantification of Drugs

The amount of temozolomide and paclitaxel was determined using the Agilent 1200 Infinity high-performance liquid 343 chromatography (HPLC) system (Agilent, Santa Clara, CA, 344 USA). Samples of the hydrogel carrying temozolomide and paclitaxel (5 mcL) were injected into Atlantis T3 C18 column (3.0×100 mm) maintained at 25° C. with a flow rate of 1.0 mL/min. Mobile phase A had acetonitrile and mobile phase B had water. Mobile phase A is maintained at 90% for 5 min and decreased to 10% for the next 5 min. Mobile phase A is kept at 10% for 3 min and increased to 90% for the next 0.1 min. Mobile phase A at 90% is maintained for the next 4.9 min. Temozolomide was detected at 280 nm and eluted at 4.2 min. Paclitaxel was detected at 260 nm and eluted at 12.4 min.

Standard curves were created using concentrations of 100, 50, 10, 5, and 1 mcg/mL for each of the eluents. The standard curve was plotted for both paclitaxel (R²=0.9996) and temozolomide (R²=0.9989).

### Gelation Temperature and Rheologic Pattern

The DVNext Wells-Brookfield Cone/Plate Rheometer is used to measure a torque meter proportional to the shear stress in sols or gels. The stationary plate is filled with 0.5 to 2 mL of sols and the working temperature increases from 10 to 50° C. A torque meter in the samples at 10-50° C. temperature range is measured by increasing from 10 to 50° C. and decreasing from 50 to 10° C. The absolute centipoise (mPa s) is then calculated, and rheologic pattern of each sample is curved.

### Drug Release Profile and Adhesion Property

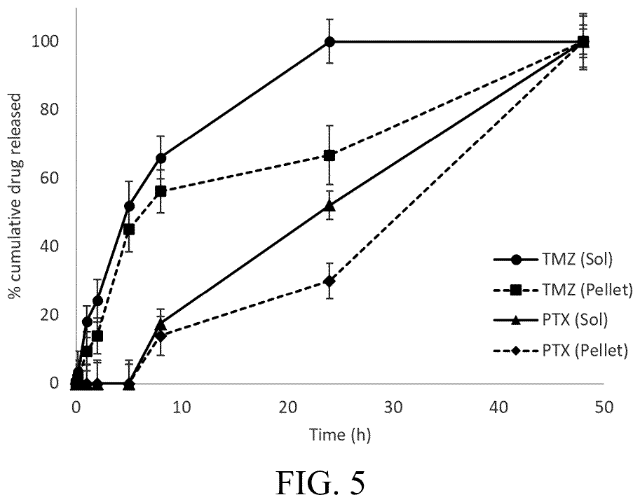

Sol (30 mcL) and pellet (6 mg/unit) forms of hydrogels carrying TMZ and paclitaxel were put into dialysis tubes (MWCO 20000) and tubes were placed in 20 mL of phosphate buffer (pH 7.2) at 37° C. with stirring. At various time points, 0, 10 min, 1, 2, 5, 8, and 24, and 48 h, the buffer was collected, and the amount of drug released was quantified by HPLC analysis. Release rate of TMZ was more rapid than that of paclitaxel. In general, drug release rate was more rapid from sol form of hydrogel than pellet form of hydrogel (FIG. 5).

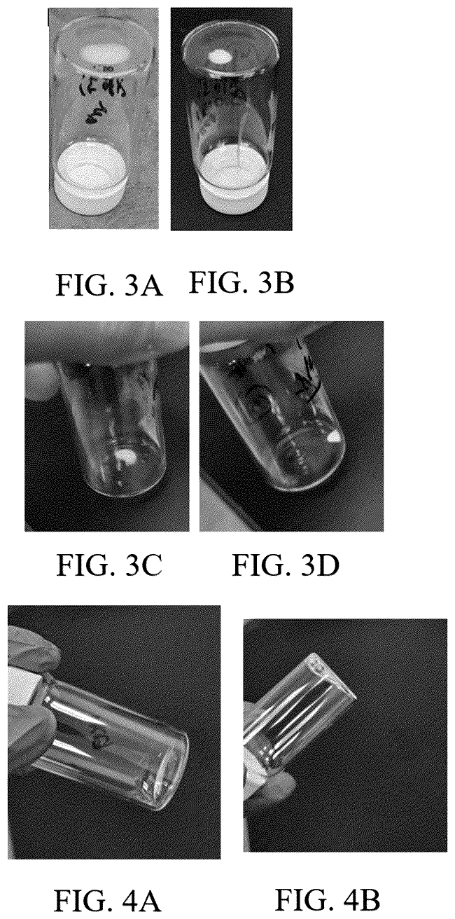

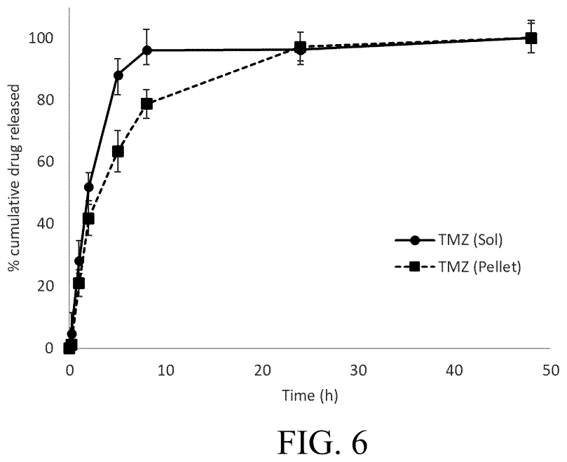

Sol (30 mcL) and pellet (6 mg/unit) forms of hydrogels carrying TMZ were put into dialysis tubes (MWCO 20000) and tubes were placed in 20 mL of phosphate buffer (pH 7.2) at 37° C. with stirring. At various time points, 0, 10 min, 1, 2, 5, 8, and 24, and 48 h, the buffer was collected and the amount of drug released was quantified by HPLC analysis. TMZ release rate was more rapid from sol form of hydrogel than pellet form of hydrogel (FIG. 6). TMZ release rate was more rapid from sols containing TMZ alone compared to that from sols containing TMZ and paclitaxel (FIGS. 5 and 6). TMZ release rate was more rapid from pellets containing TMZ alone compared to that from pellets containing TMZ and paclitaxel (FIGS. 5 and 6) Sol (30 mcL) and pellet (6 mg/unit) forms of hydrogels were placed on the bottom of 5320-mL scintillation vial and added in 1 mL of phosphate buffer (pH 7.2) at 37° C. Every 5 min, the medium was replenished with 1 mL of fresh phosphate buffer for total 30 min. Both hydrogel and pellet were adherent to the glass vial for 30 min. Both absorbed c.a. 140 mcL of buffer (FIG. 3A for sol and 3B for pellet).

Sol (30 mcL) and pellet (6 mg/unit) forms of hydrogels carrying TMZ only was tested with similar method. Pellet was adherent to the glass vial for 30 min and absorbed c.a. 120 mcL of butter. Hydrogel was adherent for 20 min but lost 40% w/w in the last 10 min (FIG. 3C for sol and 3D for pellet).

### Stability of Sol and Pellet

Sol and pellet forms of hydrogels are kept in the glass scintillation vial at 20-22° C. at relative humidity 30-60%. At various time points, 0, 1, 3, 5, 7, 14, 30, 90, 180 days, samples are collected to quantify un-degraded (un-changed) drugs.

### Example 2

Addition hydrogel formulations were also made with various drugs or drugs combinations. The formulations were made according to the method described in Example 1, but with the following amount of mannitol and drugs:
10 mg mannitol and 2 mg paclitaxel;
10 mg mannitol and 2 mg rapamycin;
10 mg mannitol, 2 mg paclitaxel, and 2 mg rapamycin;
10 mg mannitol and 10 mg paclitaxel; or
10 mg mannitol, 10 mg paclitaxel, and 10 mg rapamycin.

### Example 3

This example shows method of treating glioblastoma with the hydrogel carrying TMZ, or both TMZ and paclitaxel that are prepared according to Example 1. In general, rat model of glioblastoma is generated. The xenografted tumor is removed and the pellets of the hydrogels are implanted to the surgical site. Development and/or recurrence of the tumor in the animals is tested.

For intracranial implantation of the (9 L gliosarcoma), (F344 female) rats are anesthetized. The heads are shaved with clippers and prepared with alcohol and prepiodyne solution. A midline scalp incision is made, exposing the sagittal and coronal sutures. With the use of an electric drill with a 2-mm round cutting burr, a small hole is made in the skull centered 3 mm lateral to the sagittal suture and 5 mm posterior to the coronal suture. Care is taken to avoid the sagittal sinus. Forceps are used to lift off the remaining bone. Under microscopic magnification, a dural opening and then cortical opening are made. A small area of cortex and white matter is resected, and, once hemostasis is achieved, tumor cells are placed in the resection cavity. The skin is then closed with surgical staples. A few days later the animals are re-anesthetized, and the same incision is opened. Maximum Tolerated Dose (MTD) of hydrogel with TMZ and paclitaxel (PTX) are tested in animals receiving different numbers of discs (1 pellet n=8, 2 pellet n=8, 3 discs n=8) post-surgery.

For the efficacy study, the animals are randomly separated into 8 groups (n=8 each): (1) Untreated control; (2) Empty THF; (3) Intracranial THF (TMZ); (4) Intracranial THF (TMZ+PTX); (5) Oral TMZ; (6) Oral TMZ and IV PTX; (7) Intracranial carmustine (BCNU); (8) Oral TMZ and intracranial BCNU; (9) Intracranial THF (TMZ+PTX) and radiation therapy (XRT); (10) Oral TMZ and intracranial BCNU and radiotherapy; and (11) radiotherapy.

Animals are observed daily and euthanized upon symptoms of tumor growth and increased morbidity. The experiment is continued for a few months for long term survival. Body weights of animals are recorded. Surgical and treatment procedures are recorded and saved as video clips as requested.

At the time of death or sacrifice at the end of the experiment, brains are carefully monitored to detect residual gels. Photocopies and weights of brains collected from animals are recorded. Brains and major organs are extracted for histological analysis.

### Example 4

This example describes an exemplary method of preparing and characterizing a hydrogel formulation carrying temozolomide (TMZ), paclitaxel, or both TMZ and paclitaxel for topical treatment.

PLGA-PEG-PLGA (1.5k-1.5k-1.5k Mw) triblock copolymer was dissolved in water at 200 mg/mL at 20-22° C. PLGA-PEG-PLGA (1k-1k-1k Mw) triblock copolymer was dissolved in water at 200 mg/mL at or below 10° C. In a (sterilized) glass scintillation vial, 200 mcL of PLGA-PEG-PLGA (1.5k-1.5k-1.5k) (200 mg/mL), 100 mcL of PLGA-PEG-PLGA (1k-1k-1k) (200 mg/mL), and 10 mg mannitol, were mixed with 3.7 mL of distilled water. 3 mL of acetone was added to the mixture and vortexed vigorously for 1-2 min at 20-22° C. The final mixture was then filtered through 0.2 micrometer regenerated cellulose (RC) filter and frozen at −80° C. for at least 30 min. The frozen mixture was then lyophilized (below 0.8 mbar at −45° C.) for 24 h. The lyophilized cake was then rehydrated with 200 mcL of sterile water at 20-22° C. Rehydration time was 10-60 min, depending on the formulation. Total 400 mcL of reconstituted formulation was prepared and incorporated in ointment base to make 1.5 mL ointment preparation.

PLGA-PEG-PLGA (1.5k-1.5k-1.5k Mw) triblock copolymer was dissolved in water at 200 mg/mL at 20-22° C. PLGA-PEG-PLGA (i1k-1k-1k Mw) triblock copolymer was dissolved in water at 200 mg/mL at or below 10° C. In a (sterilized) glass scintillation vial, 200 mcL of PLGA-PEG-PLGA (1.5k-1.5k-1.5k) (200 mg/mL), 100 mcL of PLGA-PEG-PLGA (i1k-1k-1k) (200 mg/mL), 10 mg mannitol, and 30 mg TMZ were mixed with 3.7 mL of distilled water. 3 mL of acetone was added to the mixture and vortexed vigorously for 1-2 min at 20-22° C. The final mixture was then filtered through 0.2 micrometer regenerated cellulose (RC) filter and frozen at −80° C. for at least 30 min. The frozen mixture was then lyophilized (below 0.8 mbar at −45° C.) for 24 h. The lyophilized cake was then rehydrated with 200 mcL of sterile water at 20-22° C. Rehydration time was 10-60 min, depending on the formulation. Total 200 mcL of reconstituted formulation (30 mg TMZ) was incorporated in ointment base to make 1.5 mL ointment preparation (2 mg TMZ per 100 mcL dose).

PLGA-PEG-PLGA (1.5k-1.5k-1.5k Mw) triblock copolymer was dissolved in water at 200 mg/mL at 20-22° C. PLGA-PEG-PLGA (i1k-1k-1k Mw) triblock copolymer was dissolved in water at 200 mg/mL at or below 10° C. In a (sterilized) glass scintillation vial, 200 mcL of PLGA-PEG-PLGA (1.5k-1.5k-1.5k) (200 mg/mL), 100 mcL of PLGA-PEG-PLGA (i1k-1k-1k) (200 mg/mL), 10 mg mannitol, and 30 mg TMZ were mixed with 3.7 mL of distilled water. 3 mL of acetone was added to the mixture and vortexed vigorously for 1-2 min at 20-22° C. The final mixture was then filtered through 0.2 micrometer regenerated cellulose (RC) filter and frozen at −80° C. for at least 30 min. The frozen mixture was then lyophilized (below 0.8 mbar at −45° C.) for 24 h. The lyophilized cake was then rehydrated with 200 mcL of sterile water at 20-22° C. Rehydration time was 10-60 min, depending on the formulation. Total 400 mcL of reconstituted formulation (60 mg TMZ) was prepared and incorporated in ointment base to make 1.5 mL ointment preparation (4 mg TMZ per 100 mcL dose).

PLGA-PEG-PLGA (1.5k-1.5k-1.5k Mw) triblock copolymer was dissolved in water at 200 mg/mL at 20-22° C. PLGA-PEG-PLGA (i1k-1k-1k Mw) triblock copolymer was dissolved in water at 200 mg/mL at or below 10° C. In a (sterilized) glass scintillation vial, 200 mcL of PLGA-PEG-PLGA (1.5k-1.5k-1.5k) (200 mg/mL), 100 mcL of PLGA-PEG-PLGA (i1k-1k-1k) (200 mg/mL), 10 mg mannitol, and 30 mg TMZ were mixed with 3.7 mL of distilled water. Paclitaxel dissolved in acetone at 11.2 mg/3 mL, and the 3 mL acetone with 11.2 mg paclitaxel was added to the mixture and vortexed vigorously for 1-2 min at 20-22° C. The final mixture was then filtered through 0.2 micrometer regenerated cellulose (RC) filter and frozen at −80° C. for at least 30 min. The frozen mixture was then lyophilized (below 0.8 mbar at −45° C.) for 24 h. The lyophilized cake was then rehydrated with 200 mcL of sterile water at 20-22° C. Rehydration time was 10-60 min, depending on the formulation. Total 400 mcL of reconstituted formulation (60 mg TMZ and 22.5 mg PTX) was prepared and incorporated in ointment base to make 1.5 mL ointment preparation (4 mg TMZ and 1.5 mg PTX per 100 mcL dose).

### Example 5

This example shows an exemplary method of treating melanoma with formulations prepared in Example 4.

Melanoma xenograft mouse model is used for testing the efficacy of hydrogel formulations with TMZ or TMZ/paclitaxel combination. Melanoma cell lines are injected subcutaneously to the mice. When sufficient tumors reach a tumor volume approximating 4-10 mm³, animals are randomized and placed onto study. Randomization range will be confirmed in protocol.

Animals have hair on tumor and surrounding area removed prior to application of the formulations. The formulations are supplied in a syringe and applied using a plastic spatula. Animals are separated from each other for approximately 1-2 hours after application of formulations to allow for absorption.

The animal groups and study plan are summarized in the table 1 below.

TABLE 1

| | | | Dose | Dose | Dose | Dose | | |
|---|---|---|---|---|---|---|---|---|
| Group | N | Treatment | Amount | ROA | Vol | Schedule | BW | TVM |
| 1 | 8 | Vehicle | 0 | Topical | 100 | QD × 8, then | BIW | Calipers |
| | | | | | mcL | TIW × 2 wks | | BIW |
| 2 | 8 | Article # 2 | 4 mg API | Topical | 100 | QD × 8, then | | |
| | | (Ointment) | | | mcL | TIW × 2 wks | | |
| 3 | 8 | Article # 4 | 4 mg/1.5 | Topical | 100 | QD × 8, then | | |
| | | (Ointment) | mg APIs | | mcL | TIW × 2 wks | | |
| 4 | 8 | Article #1 | 4 mg API | Topical | 100 | BID × 8, then | | |
| | | (Ointment) | | | mcL | TIW × 2 wks | | |

QDx8, then TIW×2 wks indicates treatment is given every day for the first 8 days, and then 3 times a week for 2 weeks. BID×8, then TIW×2 wks indicates that treatment is given twice a day for the first 8 days, and then 3 times a week for 2 weeks. BIW indicates body weight is measured twice a week. Calipers BIW indicates that tumor volumes are measured with calipers twice a week.

### Example 6

This example shows exemplary formulations and methods for treating melanoma.

Two PLGA-b-PEG-b-PLGA triblock copolymers (Polyscitech, West Lafayette, IN), PLGA_{1,000}-b-PEG_{1,000}-b-PLGA_{1,000} (LA:GA 50:50) and PLGA_{1,500}-b-PEG_{1,500}-b-PLGA_{1,500} (LA:GA 50:50), and TMZ (Selleckchem, Houston, TX) were dissolved in DI water at 1:1.9 w/w ratio along with mannitol (mannitol:TMZ=1:3 w/w) at 25° C. PTX (LC Laboratories, Woburn, MA) dissolved in acetone (Fisher Scientific, Hampton, NH) was then added to the polymer/TMZ mixture and vigorously vortex for 5 min. The mixture was stored at −40° C. for 1 h and lyophilized at −52° C. under 0.024 mBar (FreeZone 2.5; Labconco, Kansas City, MO) for 24 h. The lyophilized cake was rehydrated with DI water at 25° C. to prepare hydrogels that contain PLGA_{1,500}-b-PEG_{1,500}-b-PLGA_{1,500} and PLGA_{1,000}-b-PEG_{1,000}-b-PLGA_{1,000} at 2:1 w/w ratio. To prepare hydrogels creams, 5 mL of rehydrated hydrogels were mixed with aquaphor (Beiersdorf Inc., Stamford, CT) to yield c.a. 10 mL of hydrogel creams, yielding 4 mg TMZ or 4 mg/1.5 mg TMZ/PTX per 100 mL of hydrogel creams.

The content of drugs was quantified using Reverse Phase HPLC (RP-HPLC) analysis with an Agilent 1220 Infinity LC system (Shimadzu, Japan). Samples (5 μL) were injected into Eclipse C18 column (3.0×150 mm, 3.5 μm, Agilent, Santa Clara, CA). The flow rate was 0.8 mL/min, and the column was kept at 25° C. The separation of PTX and TMZ was carried out in a gradient mode using acetonitrile (Fisher Scientific, Hampton, NH) with 0.1% trifluoroacetic acid (TFA; Sigma-Aldrich, St. Louis, MO) as the mobile phase A and water with 0.1% TFA as the mobile phase B. The gradient began with 90% mobile phase A for 5 min and then decreased from 90 to 10% mobile phase A in 5 min and held at 10% for 3 min. The gradient went up from 10 to 90% mobile phase A in the last 5 min. PTX and TMZ were monitored at 260 nm and eluted at 15 min and 0.8 min, respectively. The area under the curves representing PTX and TMZ were used to quantify the amounts of drugs in each sample.

Z-average diameters of hydrogels were determined by dynamic light scattering (DLS) measurements using ZetaSizer Nano ZS (Malvern Instruments, United Kingdom) at 10, 25, or 37° C. with a detection angle of 1730 and a He—Ne ion laser (4 mW, λmax=633 nm) for the incident beam (19). The scattering intensity was detected using a sensitive avalanche photodiode detector (APD) and analyzed with a digital autocorrelator which generated a correlation function based on the cumulant analysis using the Stokes-Einstein equation. Prior to the particle size detection, each sample was diluted to obtain a 3% total polymer level.

The cone rheometer (DVNext Wells-Brookfield Cone Rheometer; AMETEK Brookfield Middleboro, MA) was used to measure a torque meter at discrete rotational speeds. This torque measuring system consisted of a calibrated beryllium-copper spring connecting the drive mechanism to a rotating cone, and senses the resistance to rotation caused by the presence of a sample between the cone and a stationary flat plate. Two milliliters of test articles were placed in the cone. The test was carried out at 25° C. and 37° C. and the cone was rotated at 10 rpm. The readings for each sample were made after 5 min. For gelation temperature measurements, each sample stored in a glass scintillation vial was placed on the hot plate. The temperature of the plate increased from 25 to 40° C. and each vial was gently swirled. The temperature that allowed the sample to be inverted without dropping/sliding down on the wall (formation of a gel) was recorded.

The viscosity of empty hydrogel (vehicle) with 16% w/v of polymer level at 25° C. was 13.08±2.26 cP. By adjusting temperature to 37° C., the viscosity of empty hydrogel increased to 34.20±3.25 cP. The increase of temperature from 25° C. to 37° C. caused a 2-fold increase of the z-average particle size of empty hydrogel from 125.2±5.8 nm to 299.2±88.4 nm. The viscosity and z-average particle size of TMZ hydrogel at 25° C. were 41.20±3.71 cP and 141.6±17.3 nm, respectively. Those at 37° C. showed approximately 2-fold increases, which were 91.56±4.46 cP and 353.0±34.8 nm, respectively. The gelation temperature of hydrogel carrying TMZ was 33.9±1.5° C., which showed a slight decrease in comparison to that of empty hydrogel. The viscosity of hydrogel carrying PTX/TMZ was the highest, 68.67±5.63 and 125.6±6.92 at 25° C. and 37° C., respectively. The increase of temperature from 25° C. to 37° C. caused a 4-fold increase of the z-average particle size of PTX/TMZ hydrogel from 96.3±9.3 nm to 426.8±26.1 nm. The gelation temperature of hydrogel carrying PTX/TMZ was the lowest, 32.8±1.1° C. Particle size distribution, viscosity, and gelation temperature of PTX/TMZ formulation, TMZ formulation, and empty vehicle are shown below in Table 2

TABLE 2

| | | | | | Gelation |
|---|---|---|---|---|---|
| | Particle size (nm) | | | | temperature |
| Properties | PDI | | Viscosity (cP) | | (° C.) |
| Temperature | 25° C. | 37° C. | 25° C. | 37° C. | |
| PTX/TMZ | 96.3 ± 9.3 | 426.8 ± 26.1 | 68.67 ± 5.63 | 125.6 ± 6.92 | 32.8 ± 1.1 |
| hydrogel | 0.569 ± 0.166 | 0.914 ± 0.042 | | | |
| TMZ hydrogel | 141.6 ± 17.3 | 353.0 ± 34.8 | 41.20 ± 3.71 | 91.56 ± 4.46 | 33.9 ± 1.5 |
| | 0.479 ± 0.046 | 0.849 ± 0.262 | | | |
| Empty | 125.2 ± 5.8 | 299.2 ± 88.4 | 13.08 ± 2.26 | 34.20 ± 3.25 | 35.5 ± 2.3 |
| hydrogel | 0.557 ± 0.284 | 0.683 ± 0.106 | | | |
| (vehicle) | | | | | |

Cryogenic vials containing B16-F10 murine melanoma tumor cells were thawed and cultured according to the manufacturer's protocol. On the day of injection, cells were washed in serum-free media, counted, and resuspended in cold serum-free media at a concentration of 200,000 viable cells/100 μL. Cells were prepared for injection by withdrawing 100 μL of the cell suspension into a 1 mL syringe. C57BL/6 mice at 6-9 weeks old (The Jackson Laboratory, Bar Harbor, ME) were prepared for injection using standard approved anesthesia. Seventeen C57BL/6 mice were shaved and 100 μL of the cell suspension was subcutaneously injected into the rear flank of the animals.

Animals were monitored weekly for palpable tumors, or any changes in appearance or behavior. Daily monitoring was taken place for mice showing any signs of morbidity or mortality. Once tumors were palpable, tumors were measured at least once a week using calipers. Tumor volume was calculated using the following equation: (longest diameter×shortest diameter²)/2. Once tumors were of the appropriate size to begin the study, tumors were measured at least 2 times per week for the duration of the study. When the average tumor volume reached 80-120 mm³, mice were randomly assigned to the respective treatment groups (empty hydrogel cream, n=5; TMZ hydrogel cream, n=6; PTX/TMZ hydrogel cream, n=6) and dosed within 24 h of randomization. The tumor and surrounding area were shaved to ensure the proper application of the test articles. Test articles (empty hydrogel cream, TMZ (4 mg per dosing) hydrogel cream, and PTX/TMZ (4/1.5 mg per dosing) hydrogel cream) were stored in 3-mL sterile syringes. One hundred μL of each test article was extruded for each dosing. The test articles were applied on the tumor site by gently rubbing it onto the skin using a cotton tip. The test articles were applied topically every day for 8 days and thereafter 3 times a week for a week. Body weight was measured at least 2 times a week following randomization and initiation of treatment. Body weight loss were calculated based on the body weight of the mouse on the first day of treatment. The animal was euthanized if body weight loss of >20% was observed. Dosing holiday and/or nutritional supplements were provided to the animal based on the study director's assessment of animal health. If there were no signs of recovery, the animal was sacrificed for humane reasons as per our IACUC protocol regulations. Clinical observations were performed at least 2 times a week at the time of tumor and body weight measurements. During routine monitoring, the animals were checked for any adverse effects of tumor growth and treatments on behavior such as mobility, food and water consumption, eye/hair matting and any other abnormalities. Mortality and observed abnormal clinical signs were recorded for individual animals in detail. Daily monitoring was taken place for mice showing severe signs of pain or distress and any moribund animals were euthanized the same day that they were recognized as moribund.

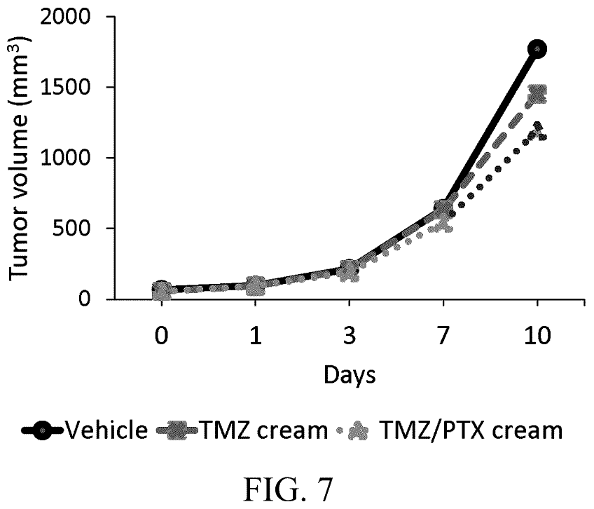

FIG. 7 shows TMZ hydrogel cream and TMZ/PTX hydrogel cream exhibited inhibitory effects on the tumor growth in the melanoma mouse model, with the TMZ/PTX hydrogel cream being more effective than the single TMZ hydrogel cream.

### Example 7

This example shows method of treating brain tumor with the hydrogel carrying TMZ that were prepared according to Example 1. In general, rat model of glioblastoma was generated with 9 L gliosarcoma cell. The survival of the animals after tumor implantation was measured.

For intracranial implantation of the (9 L gliosarcoma), (F344 female) rats were anesthetized. The heads were shaved with clippers and prepared with alcohol and prepiodyne solution. A midline scalp incision was made, exposing the sagittal and coronal sutures. With the use of an electric drill with a 2-mm round cutting burr, a small hole was made in the skull centered 3 mm lateral to the sagittal suture and 5 mm posterior to the coronal suture. Care was taken to avoid the sagittal sinus. Forceps were used to lift off the remaining bone. Under microscopic magnification, a dural opening and then cortical opening were made. A small area of cortex and white matter was resected, and, once hemostasis is achieved, tumor cells were placed in the resection cavity. The skin was then closed with surgical staples. A few days later the animals are re-anesthetized, and the same incision is opened.

The animals were randomly separated into 8 groups (n=8 each): (1) Untreated control; (2) Empty THF; (3) oral TMZ (50 mg/kg for 5 days); and (4) Intracranial pellet with TMZ (4 mg TMZ per pellet).

Animals were observed daily and euthanized upon symptoms of tumor growth and increased morbidity. The experiment was continued for more than 40 days. Body weights of animals were recorded.

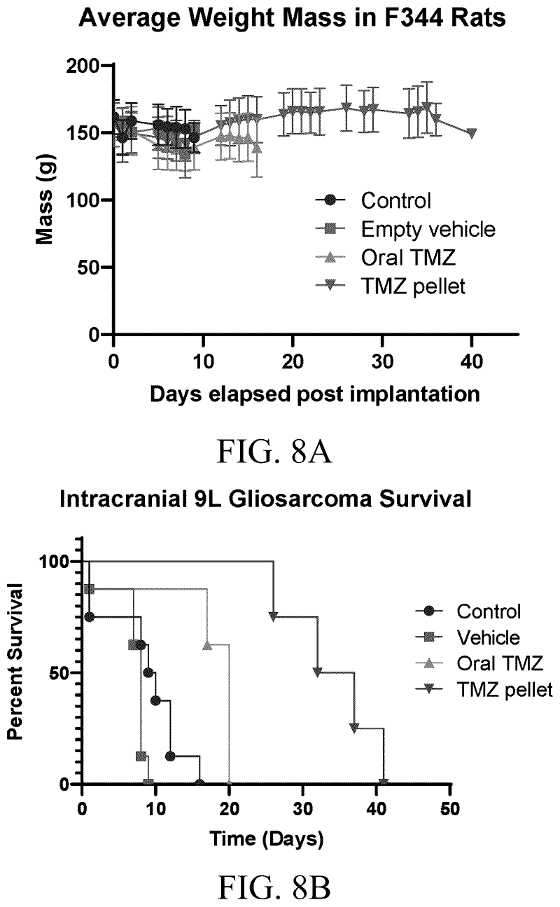

FIG. 8A shows the body weights of the animals during the experiment, which suggest that the pellet with TMZ did not have an effect on body weight change than the control group. FIG. 8B shows that the treatment with the hydrogel carrying TMZ significantly prolonged animals survival compared to the non-treated groups and the oral TMZ treated group, suggesting an inhibitory effects of the pellet with TMZ on brain tumor.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A composition comprising:
a first polymer and a second polymer; and
one or more therapeutic agents,
wherein:
the first and the second polymers are PLGA-PEG-PLGA triblock copolymers,
the first polymer has a molecular weight higher than the second polymer,
a molar ratio of the first polymer to the second polymer is from 3:1 to 1:1, and
a ratio of a total weight of the one or more therapeutic agents to a total weight of the first and second polymers is at least 2%.

2-5. (canceled)

6. The composition of claim 1, wherein the ratio of the total weight of the one or more therapeutic agents to the total weight of the first and the second polymers is from 30% to 145%.

7. The composition of claim 1, further comprising water, wherein a ratio of a weight of the water to a total weight of other components in the composition is from 80:1 to 15:1.

8. (canceled)

9. The composition of claim 1, wherein the molar ratio between the first and the second polymers is about 2:1.

10. (canceled)

11. The composition of claim 1, wherein the composition is a non-flowing thermosensitive hydrogel at a temperature from 28° C. to 50° C. and a free-flowing solution at a temperature from 0° C. to 26° C.

12. The composition of claim 1, wherein the composition is in a solid form, and the solid form becomes a hydrogel when mixing with water or an aqueous solution from about 20% g/mL to 70% g/mL measured as total weight of the solid form by volume of water.

13. The composition of claim 1, further comprising a bulking agent, and wherein a ratio of the total weight of the first polymer, the second polymer and the therapeutic agents to a weight of the bulking agent is at least 9.

14-15. (canceled)

16. The composition of claim 1, wherein the one or more therapeutic agents comprises one or more hydrophobic chemotherapeutic drugs.

17. (canceled)

18. The composition of claim 16, wherein the one or more hydrophobic chemotherapeutic drugs comprises a taxane compound.

19. The composition of claim 18, wherein the taxane compound is paclitaxel or an analog thereof.

20. (canceled)

21. The composition of claim 19, wherein a ratio of a weight of the paclitaxel or an analog thereof to the total weight of the first and the second polymers is from 1.5% to 43%.

22. The composition of claim 16, wherein the one or more chemotherapeutic drugs comprises an imidazotetrazine derivative compound.

23. The composition of claim 22, wherein the imidazotetrazine derivative compound is temozolomide or an analog thereof.

24. (canceled)

25. The composition of claim 23, wherein a ratio of a weight of the temozolomide or an analog thereof to the total weight of the first and the second polymers is from 30% to 100%.

26-27. (canceled)

28. The composition of claim 1, which comprises two therapeutic agents, wherein the two therapeutic agents are an imidazotetrazine derivative compound and a taxane compound.

29-30. (canceled)

31. The composition of claim 28, wherein
a. a ratio of the total weight of a weight of the taxane compound to the total weight of the first and the second polymers is from 1.5% to 43%, and
b. a ratio of a weight of the imidazotetrazine derivative compound to the total weight of the first and the second polymers is 30% to 100%.

32. (canceled)

33. The composition of claim 1, which is in a form of a pellet.

34-35. (canceled)

36. The composition of claim 1, further comprising an ointment base.

37. (canceled)

38. A method of treating a disease, the method comprising administering the composition of claim 1 to a subject in need thereof.

39-48. (canceled)

49. A method of producing a composition comprising:
(a) mixing a first polymer, a second polymer, and one or more therapeutic agents in water;
(b) mixing an organic solvent with the mixture from (a); and
(c) lyophilizing the mixture from (b),
wherein
the first and the second polymers are PLGA-PEG-PLGA triblock copolymers,
the first polymer has a molecular weight higher than the second polymer,
a molar ratio of the first polymer to the second polymer is from 3:1 to 1:1, and
a ratio of a weight of the one or more therapeutic agents to a total weight of the first and second polymers is at least 30%.

50-57. (canceled)
